(19) United States
(12) Patent Application Publication
(10) Pub. No.: US 2018/0223363 A1
(43) Pub. Date: Aug. 9, 2018

(54) BIOMARKERS FOR HBV TREATMENT RESPONSE

(71) Applicant: Hoffmann-La Roche Inc., Little Falls, NJ (US)

(72) Inventors: Lore Gruenbaum, Cos Cob, CT (US); Hua He, London (GB); Vedran Pavlovic, London (GB); Cynthia Wat, London (GB)

(73) Assignee: Hoffmann-La Roche Inc., Little Falls, NJ (US)

(21) Appl. No.: 15/869,431

(22) Filed: Jan. 12, 2018

Related U.S. Application Data
(63) Continuation of application No. PCT/EP2016/066460, filed on Jul. 12, 2016.

(30) Foreign Application Priority Data
Jul. 15, 2015 (EP) 15176790.2

(51) Int. Cl. C12Q 1/6883; A61K 38/21; A61P 31/20; A61K 47/60

(52) U.S. Cl. CPC C12Q 1/6883; A61K 38/212; C12Q 2600/106; A61K 47/60; C12Q 2600/156; A61P 31/20

(57) ABSTRACT

The present invention relates to methods that are useful for predicting the response of hepatitis B virus (HBV) infected patients to pharmacological treatment.

### CROSS-REFERENCE TO RELATED APPLICATIONS

This application is a continuation of International Patent Application No. PCT/EP2016/066460, having an international filing date of Jul. 12, 2016, the entire contents of which are incorporated herein by reference, and which claims benefit under 35 U.S.C. § 119 to European Patent Application No. 15176790.2, filed on Jul. 15, 2015.

### FIELD OF THE INVENTION

The present invention relates to methods that are useful for predicting the response of hepatitis B virus (HBV) infected patients to pharmacological treatment.

### BACKGROUND OF THE INVENTION

The hepatitis B virus (HBV) infects 350-400 million people worldwide; one million deaths resulting from cirrhosis, liver failure, and hepatocellular carcinoma due to the infection are recorded annually. The infecting agent, hepatitis B virus (HBV), is a DNA virus which can be transmitted percutaneously, sexually, and perinatally. The prevalence of infection in Asia (≥8%) is substantially higher than in Europe and North America (<2%) (Dienstag J. L., Hepatitis B Virus Infection., N. Engl. J. Med. 2008; 359: 1486-1500). The incidence of HBV acquired perinatally from an infected mother is much higher in Asia, leading to chronic infection in >90% of those exposed (WHO Fact Sheet No 204; revised August 2008). Additionally, 25% of adults who become chronically infected during childhood die from HBV-related liver cancer or cirrhosis (WHO Fact Sheet No 204; revised August 2008). Interferon alpha (IFNa) is a potent activator of anti-viral pathways and additionally mediates numerous immuno-regulatory functions (Muller U., Steinhoff U., Reis L. F. et al., Functional role of type I and type II interferons in antiviral defense, Science 1994; 264: 1918-21).

The efficacy of PEGASYS® (Pegylated IFN alfa 2a 40KD, Peg-IFN) at a dose of 180 μg/week in the treatment of HBV was demonstrated in two large-scale pivotal studies. One study was in HBeAg-negative patients (WV16241) and the other in HBeAg-positive patients (WV16240).

WV16241 was conducted between June 2001 and August 2003; 552 HBeAg-negative CHB patients were randomized to one of three treatment arms: PEG-IFN monotherapy, PEG-IFN plus lamivudine or lamivudine alone for 48 weeks. Virologic response (defined as HBV DNA <20,000 copies/mL) assessed 24 weeks after treatment cessation was comparable in the groups that received PEG-IFN (43% and 44%) and both arms were superior to the lamivudine group (29%) (Marcellin P., Lau G. K., Bonino F. et al., Peginterferon alfa-2a alone, lamivudine alone, and the two in combination in patients with HBeAg-negative chronic hepatitis B, N. Engl. J. Med. 2004; 351: 1206-17).

Study WV16240 was conducted between January 2002 and January 2004. In this study, 814 HBeAg-positive CHB patients were randomized to the same treatment arms as in WV16241, i.e. PEG-IFN monotherapy, PEG-IFN plus lamivudine or lamivudine alone for 48 weeks. Responses assessed 24 weeks after treatment cessation showed a 32% rate of HBeAg seroconversion in the PEG-IFN monotherapy group compared to 27% and 19% with PEG-IFN+lamivudine and lamivudine monotherapy respectively (Lau G. K., Piratvisuth T,. Luo K. X. et al., Peginterferon Alfa-2a, Lamivudine, and the Combination for HBeAg-Positive Chronic Hepatitis B, N. Engl. J. Med. 2005; 352: 2682-95). Metaanalysis of controlled HBV clinical studies has demonstrated that PEG-IFN-containing treatment facilitated significant HBsAg clearance or seroconversion in CHB patients over a lamivudine regimen (Li W. C., Wang M. R., Kong L. B. et al., Peginterferon alpha-based therapy for chronic hepatitis B focusing on HBsAg clearance or seroconversion: a meta-analysis of controlled clinical trials, BMC Infect. Dis. 2011; 11: 165-177).

More recently, the Neptune study (WV19432) was conducted between May 2007 and April 2010 and compared PEG-IFN administered as either 90 or 180 μg/week administered over either 24 or 48 weeks in HBeAg-positive patients (Liaw Y. F., Jia J. D., Chan H. L. et al., Shorter durations and lower doses of peginterferon alfa-2a are associated with inferior hepatitis B e antigen seroconversion rates in hepatitis B virus genotypes B or C, Hepatology 2011; 54: 1591-9). Efficacy was determined at 24 weeks following the end of treatment. This study, demonstrated that both the lower dose and shorter durations of treatment were inferior to the approved dose and duration previously used in the WV16240 study, thus confirming that the approved treatment regimen of i.e. 180 μg/week for 48 weeks is the most beneficial for patients with HBeAg-positive CHB.

However, despite the fact that PEG-IFN has been successfully used in the treatment of CHB, little is known of the impact of host factors (genetic and non-genetic) and viral factors on treatment response.

Although viral and environmental factors play important roles in HBV pathogenesis, genetic influence is clearly present. While small genetic studies have suggested the possible implications of host immune/inflammation factors (e.g. HLA, cytokine, inhibitory molecule) in the outcomes of HBV infection, a genome-wide association study (GWAS) clearly demonstrated that 11 single nucleotide polymorphisms (SNPs) across the human leukocyte antigen (HLA)-DP gene region are significantly associated with the development of persistent chronic hepatitis B virus carriers in the Japanese and Thai HBV cohorts (Kamatani Y., Wattanapokayakit S., Ochi H. et al., A genome-wide association study identifies variants in the HLA-DP locus associated with chronic hepatitis B in Asians. Nat. Genet. 2009; 41: 591-595). Subsequently this finding was also confirmed in a separate Chinese cohort study using a TaqMan based genotyping assay (Guo X., Zhang Y., Li J. et al., Strong influence of human leukocyte antigen (HLA)-DP gene variants on development of persistent chronic hepatitis B virus carriers in the Han Chinese population, Hepatology 2011; 53: 422-8). Furthermore, a separate GWAS and replication analysis concluded similar results that there is significant association between the HLA-DP locus and the protective effects against persistent HBV infection in Japanese and Korean populations (Nishida N., Sawai H., Matsuura K. et al., Genome-wide association study confirming association of HLA-DP with protection against chronic hepatitis B and viral clearance in Japanese and Korean. PLos One 2012; 7: e39175). Finally, two additional SNPs (rs2856718 and rs7453920) within the HLA-DQ locus were found to have an independent effect of HLA-DQ variants on CHB susceptibility (Mbarek H., Ochi H., Urabe Y. et al., A genome-wide association study of chronic hepatitis B identified novel risk locus in a Japanese population, Hum. Mol. Genet. 2011; 20: 3884-92). Taken together, robust genetic evidence suggests that in the Asian population, polymorphic variations at the HLA region contribute significantly to the progression of chronic hepatitis B following acute infection in Asian populations.

Meta-analysis of controlled HBV clinical trials has demonstrated that conventional IFN alfa-or pegylated IFN alfa (2a or 2b)-containing treatment facilitated significant HBsAg clearance or seroconversion in CHB patients over lamivudine regimens (Li W. C., Wang M. R., Kong L. B. et al., Peginterferon alpha-based therapy for chronic hepatitis B focusing on HBsAg clearance or seroconversion: a meta-analysis of controlled clinical trials, BMC Infect. Dis. 2011; 11: 165-177). However, despite the fact that Peg-IFN has been successfully used in the treatment of CHB, little is known regarding the relationship between treatment response and the impact of host factors at the level of single nucleotide polymorphisms (SNPs). Pegylated interferon alfa, in combination with ribavirin (RBV) has been successfully used in the treatment of chronic hepatitis C virus (HCV) infection. A major scientific finding in how HCV patients respond to Peg-IFN/RBV treatment is that via genome-wide association studies (GWAS), genetic polymorphisms around the gene IL28B on chromosome 19 are strongly associated with treatment outcome (Ge D., Fellay J., Thompson A. J. et al., Genetic variation in IL28B predicts hepatitis C treatment-induced viral clearance, Nature 2009; 461: 399-401; Tanaka Y., Nishida N., Sugiyama M. et al., Genome-wide association of IL28B with response to pegylated interferon-alpha and ribavirin therapy for chronic hepatitis C, Nat. Genet. 2009; 41: 1105-9; Suppiah V., Moldovan M., Ahlenstiel G. et al., IL28B is associated with response to chronic hepatitis C interferon-alpha and ribavirin therapy, Nat. Genet. 2009; 41: 1100-4). IL28B encoded protein is a type III IFN (IFN-λ3) and forms a cytokine gene cluster with IL28A and IL29 at the same chromosomal region. IL28B can be induced by viral infection and has antiviral activity. However, in CHB patients treated with Peg-IFN, there are limited and somewhat conflicting data on the association of specific SNPs (e.g. rs12989760, rs8099917, rs12980275) around IL28B region with treatment responses (Lampertico P., Vigano M., Cheroni C. et al., Genetic variation in IL28B polymorphism may predict HBsAg clearance in genotype D, HBeAg negative patients treated with interferon alfa, AASLD 2010; Mangia A., Santoro R., Mottola et al., Lack of association between IL28B variants and HBsAg clearance after interferon treatment, EASL 2011; de Niet A., Takkenberg R. B., Benayed R. et al., Genetic variation in IL28B and treatment outcome in HBeAg-positive and -negative chronic hepatitis B patients treated with Peg interferon alfa-2a and adefovir, Scand. J. Gastroenterol. 2012, 47: 475-81; Sonneveld M. J., Wong V. W., Woltman A. M. et al., Polymorphisms near IL28B and serologic response to peginterferon in HBeAg-positive patients with chronic hepatitis B, Gastroenterology 2012; 142: 513-520).

IL28B genotype predicts response to pegylated-interferon (peg-IFN)-based therapy in chronic hepatitis C. Holmes et al. investigated whether IL28B genotype is associated with peg-IFN treatment outcomes in a predominantly Asian CHB cohort. IL28B genotype was determined for 96 patients (Holmes et al., IL28B genotype is not useful for predicting treatment outcome in Asian chronic hepatitis B patients treated with pegylated interferon-alpha, J. Gastroenterol. Hepatol., 2013, 28(5): 861-6). 88% were Asian, 62% were HBeAg-positive and 13% were METAVIR stage F3-4. Median follow-up time was 39.3 months. The majority of patients carried the CC IL28B genotype (84%). IL28B genotype did not differ according to HBeAg status. The primary endpoints were achieved in 27% of HBeAg-positive and 61% of HBeAg-negative patients. There was no association between IL28B genotype and the primary endpoint in either group. Furthermore, there was no difference in HBeAg loss alone, HBsAg loss, ALT normalisation or on-treatment HBV DNA levels according to IL28B genotype.

With whole blood sample collection in CHB patients who have been treated with Peg-IFN and have definite clinical outcomes, it is well justified that mechanistically understanding how host genetic factors affect treatment response and HBV disease biology will be tremendously beneficial to the future clinical practice of identifying patients who are likely to respond to Peg-IFN treatment and to the development of new HBV medicines.

### SUMMARY OF THE INVENTION

The present invention provides for methods for identifying patients who will respond to an anti-HBV treatment with anti-HBV agents, such as an interferon.

One embodiment of the invention provides methods of identifying a patient who may benefit from treatment with an anti-HBV therapy comprising an interferon, the methods comprising: determining the presence of a single nucleotide polymorphism in gene FCERJA on chromosome 1 in a sample obtained from the patient, wherein the presence of at least one A allele at rs7549785 indicates that the patient may benefit from the treatment with the anti-HBV treatment.

A further embodiment of the inventions provides methods of predicting responsiveness of a patient suffering from an HBV infection to treatment with an anti-HBV treatment comprising an interferon, the methods comprising: determining the presence of a single nucleotide polymorphism in gene FCER1A on chromosome 1 in a sample obtained from the patient, wherein the presence of at least one A allele at rs7549785 indicates that the patient is more likely to be responsive to treatment with the anti-HBV treatment.

Yet another embodiment of the invention provides methods for determining the likelihood that a patient with an HBV infection will exhibit benefit from an anti-HBV treatment comprising an interferon, the methods comprising: determining the presence of a single nucleotide polymorphism in gene FCER1A on chromosome 1 in a sample obtained from the patient, wherein the presence of at least one A allele at rs7549785 indicates that the patient has increased likelihood of benefit from the anti-HBV treatment.

Even another embodiment of the invention provides methods for optimizing the therapeutic efficacy of an anti-HBV treatment comprising an interferon, the methods comprising: determining the presence of a single nucleotide polymorphism in gene FCER1A on chromosome 1 in a sample obtained from the patient, wherein the presence of at least one A allele at rs7549785 indicates that the patient has increased likelihood of benefit from the anti-HBV treatment.

A further embodiment of the invention provides methods for treating an HBV infection in a patient, the methods comprising: (i) determining the presence of at least one A allele at rs7549785 in gene FCER1A on chromosome 1 in a sample obtained from the patient and (ii) administering an effective amount of an anti-HBV treatment comprising an interferon to said patient, whereby the HBV infection is treated.

Yet another embodiment of the present invention provides methods for predicting S-loss at >=24-week follow-up of treatment (responders vs. non-responders) of a patient infected with HBV to interferon treatment comprising: (i) providing a sample from said human subject, detecting the presence of a single nucleotide polymorphism in gene FCER1A on chromosome 1 and (ii) determining that said patient has a high response rate to interferon treatment measured as S-loss at >=24-week follow-up of treatment (responders vs. non-responders) if at least one A allele at rs7549785 is present.

In some embodiments, the interferon is selected from the group of peginterferon alfa-2a, peginterferon alfa-2b, interferon alfa-2a and interferon alfa-2b.

In some embodiments, the interferon is a peginterferon alfa-2a conjugate having the formula:

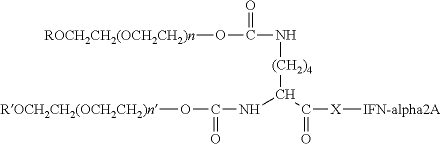

wherein R and R′ are methyl, X is NH, and n and n′ are individually or both either 420 or 520.

### BRIEF DESCRIPTION OF THE DRAWINGS

FIG. 1: Bar chart of the number of markers by chromosome in the GWAS Marker Set. Of 926,453 markers, 1,007 markers were not plotted due to unknown genomic location.

FIG. 2: Scree plot for ancestry analysis.

FIG. 3: The first two principal components of ancestry for HapMap individuals only. Population codes are as listed in Table 3.

FIG. 4: The first two principal components of ancestry for HapMap individuals; coloured according to population group (Table 3). Overlaid are patients who will be incorporated into PGx-CN-Final (black crosses) and those that will be incorporated into PGx-non-CN-Final (grey crosses).

FIG. 5: Manhattan Plots for Endpoint 1.

FIG. 6: QQ Plots for Endpoint 1.

FIG. 7: Manhattan Plots for Endpoint 2.

FIG. 8: QQ Plots for Endpoint 2.

FIG. 9: Manhattan Plots for Endpoint 3.

FIG. 10: QQ Plots for Endpoint 3.

FIG. 11: Manhattan Plots for Endpoint 4.

FIG. 12: QQ Plots for Endpoint 4.

FIG. 13: Manhattan Plots for Endpoint 5.

FIG. 14: QQ Plots for Endpoint 5.

FIG. 15: Manhattan Plots for Endpoint 6.

FIG. 16: QQ Plots for Endpoint 6.

FIG. 17: Univariate association plot under an additive model, for markers in FCER1A plus 10 kb flanking sequence.

FIG. 18: Univariate Linkage Disequilibrium (D′) analysis of markers in FCER1A.

### DETAILED DESCRIPTION OF THE INVENTION

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The terms “sample” or “biological sample” refers to a sample of tissue or fluid isolated from an individual, including, but not limited to, for example, tissue biopsy, plasma, serum, whole blood, spinal fluid, lymph fluid, the external sections of the skin, respiratory, intestinal and genitourinary tracts, tears, saliva, milk, blood cells, tumors, organs. Also included are samples of in vitro cell culture constituents (including, but not limited to, conditioned medium resulting from the growth of cells in culture medium, putatively virally infected cells, recombinant cells, and cell components).

The terms “interferon” and “interferon-alpha” are used herein interchangeably and refer to the family of highly homologous species-specific proteins that inhibit viral replication and cellular proliferation and modulate immune response. Typical suitable interferons include, but are not limited to, recombinant interferon alpha-2b such as Intron® A interferon available from Schering Corporation, Kenilworth, N.J., recombinant interferon alpha-2a such as Roferon®-A interferon available from Hoffmann-La Roche, Nutley, N.J., recombinant interferon alpha-2C such as Berofor® alpha 2 interferon available from Boehringer Ingelheim Pharmaceutical, Inc., Ridgefield, Conn., interferon alpha-n1, a purified blend of natural alpha interferons such as Sumiferon® available from Sumitomo, Japan or as Wellferon® interferon alpha-n1 (INS) available from the Glaxo-Wellcome Ltd., London, Great Britain, or a consensus alpha interferon such as those described in U.S. Pat. Nos. 4,897,471 and 4,695,623 (especially Examples 7, 8 or 9 thereof) and the specific product available from Amgen, Inc., Newbury Park, Calif., or interferon alpha-n3 a mixture of natural alpha interferons made by Interferon Sciences and available from the Purdue Frederick Co., Norwalk, Conn., under the Alferon Tradename. The use of interferon alpha-2a or alpha-2b is preferred. Interferons can include pegylated interferons as defined below.

The terms “pegylated interferon”, “pegylated interferon alpha” and “peginterferon” are used herein interchangeably and means polyethylene glycol modified conjugates of interferon alpha, preferably interferon alfa-2a and alfa-2b. Typical suitable pegylated interferon alpha include, but are not limited to, Pegasys® and Peg-Intron®.

As used herein, the terms “allele” and “allelic variant” refer to alternative forms of a gene including introns, exons, intron/exon junctions and 3′ and/or 5′ untranslated regions that are associated with a gene or portions thereof. Generally, alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides.

As used herein, the term “polymorphism” refers to the coexistence of more than one form of a nucleic acid, including exons and introns, or portion (e.g., allelic variant) thereof. A portion of a gene of which there are at least two different forms, i.e., two different nucleotide sequences, is referred to as a polymorphic region of a gene. A polymorphic region can be a single nucleotide, i.e. “single nucleotide polymorphism” or “SNP”, the identity of which differs in different alleles. A polymorphic region can also be several nucleotides long.

Numerous methods for the detection of polymorphisms are known and may be used in conjunction with the present invention. Generally, these include the identification of one or more mutations in the underlying nucleic acid sequence either directly (e.g., in situ hybridization) or indirectly (identifying changes to a secondary molecule, e.g., protein sequence or protein binding).

One well-known method for detecting polymorphisms is allele specific hybridization using probes overlapping the mutation or polymorphic site and having about 5, 10, 20, 25, or 30 nucleotides around the mutation or polymorphic region. For use in a kit, e.g., several probes capable of hybridizing specifically to allelic variants, such as single nucleotide polymorphisms, are provided for the user or even attached to a solid phase support, e.g., a bead or chip.

The single nucleotide polymorphism, “rs7549785” refers to a SNP identified by its accession number in the database of SNPs (dbSNP, www.ncbi.nlm.nih.gov/SNP/) and is located on human chromosome 1 in the FCER1A gene. FCER1A encodes the immunoglobulin epsilon (IgE) Fc receptor subunit alpha. The IgE receptor is the initiator of the allergic response. When two or more high affinity IgE receptors are brought together by allergen-bound IgE molecules, mediators such as histamine are released. The protein encoded by this gene represents the alpha subunit of the receptor.

### Abbreviations

| | |
|---|---|
| AIC | Akaike Information Criterion |
| ALT | Alanine aminotransferase |
| Anti-HBs | Antibody to hepatitis B surface antigen |
| DNA | Deoxyribonucleic acid |
| GWAS | Genome-wide Association Study |
| HAV | Hepatitis A Virus |
| HBe | Hepatitis B ‘e’ Antigen |
| HBeAg | Hepatitis B ‘e’ Antigen |
| HBV | Hepatitis B Virus |
| HCV | Hepatitis C Virus |
| HIV | Human Immunodeficiency Virus |
| HLA | Human Leucocyte Antigen |
| HWE | Hardy-Weinberg Equilibrium |
| IU/ml | International units per milliliter |
| PCA | Principal Components Analysis |
| PEGASYS | Pegylated Interferon alpha 2a 40 KD; Peg-IFN |
| Peg-IFN | Pegylated Interferon alpha 2a 40 KD; PEGASYS |
| QC | Quality Checks |
| qHBsAg | Quantitative Hepatitis B Surface Antigen |
| S-loss | Surface Antigen Loss |
| SNP | Single Nucleotide Polymorphism |
| SPC | Summary of Product Characteristics |
| TLR | Toll-like Receptor |
| Tx | Treatment |
| Vs. | Versus |

### EXAMPLES

### Objectives and Endpoints

The objective was to determine genetic variants associated with response to treatment with PEGASYS-containing regimen in patients with Chronic Hepatitis B.

The following endpoints, by patient group, were considered.

The following endpoints were considered:
1. HBe-positive patients: E-seroconversion or S-loss at >=24-week follow-up
2. HBe-positive patients: (E-seroconversion plus HBV DNA <2000 IU/ml) or S-loss at >=24-week follow-up
3. HBe-negative patients: HBV DNA <2000 IU/ml or S-loss at >=24-week follow-up
4. E-seroconversion or S-loss at >=24-week follow-up if HBe-positive and HBV DNA <2000 IU/ml or S-loss at >=24-week follow-up if HBe-negative (1 and 3)
5. (E-seroconversion plus HBV DNA <2000 IU/ml) or S-loss at >=24-week follow-up if HBe-positive and HBV DNA <2000 IU/ml or S-loss at >=24-week follow-up if HBe-negative (2 and 3)
6. S-loss at >=24-week follow-up

For all endpoints and all markers, the null hypothesis of no association, between the genotype and the endpoint, was tested against the two-sided alternative that association exists.

### Study Design

A cumulative meta-analysis, of data from company-sponsored clinical trials, and data from patients in General Practice care, is in progress. The combined data will, at the final analysis, comprise up to 1669 patients who have been treated with Pegasys for at least 24 weeks, with or without a nucleotide/nucleoside analogue, and with 24 weeks of follow-up data available.

The following trials/ patient sources were considered for inclusion:
RGT (ML22266)
S-Collate (MV22009)
SoN (MV22430)
Switch (ML22265)
Combo
New Switch (ML27928)
NEED
Italian cohort of PEG.Be.Liver
Professor Teerha (Thailand): clinical practice patients and some legacy Ph3 patients
Professor Hongfei Zhang (Beijing, China): clinical practice patients and some legacy Ph3 patients
Professor Yao Xie (Beijing, China): clinical practice patients
Professor Xin Yue Chen (Beijing, China): clinical practice patients

Adult patients with chronic hepatitis B (male or female patients >18 years of age) must meet the following criteria for study entry:
Previously enrolled in a Roche study and treated for chronic hepatitis B for at least 24 weeks with Peg-IFN±nucleoside analogue (lamivudine or entacavir) or Peg-IFN±nucleotide analogue (adefovir) with ≥24-week post-treatment follow-up or;
Treated in general practice for chronic hepatitis B with Peg-IFN according to standard of care and in line with the current summary of product characteristics (SPC)/local labeling who have no contra-indication to Peg-IFN therapy as per the local label and have been treated with Peg-IFN for at least 24 weeks and have ≥24-week post-treatment response available at the time of blood collection.
Patients are not infected with HAV, HCV, or HIV
Patients should have the following medical record available (either from historical/ongoing study databases or from medical practice notes):
Demographics (e.g. age, gender, ethnic origin)
Pre-therapy HBeAg status, known or unknown HBV genotype
Quantitative HBV DNA by PCR Test in IU/ml over time (e.g. baseline, on-treatment: 12- and 24-week, post-treatment: 24-week)
Quantitative HBsAg test (if not available, qualitative HBsAg test) and anti-HBs over time (e.g. baseline, on-treatment: 12- and 24-week, post-treatment: 24-week)
Serum ALT over time (e.g. baseline, on-treatment: 12- and 24-week, post-treatment: 24-week)

It is noted that all patients will have received active regimen.

### Analysis Populations

The majority of patients will be from China. For the purposes of statistical analysis, four analysis populations were defined as follows:
PGx-FAS is all patients with at least one genotype
PGx-GT is the subset of PGx-FAS whose genetic data passes quality checks
PGx-CN is the subset of PGx-GT who share a common genetic background in the sense that they cluster with CHB and CHD reference subjects from HapMap version3 (see below)
PGx-non-CN is the remainder of PGx-GT who do not fall within PGx-CN

Additional suffices are appended as HBePos or HBeNeg for the HBe-Positive and HBe-Negative subsets respectively, and as interim1,. . . interim3, and final, according to the stage of the analysis.

### Genetic Markers

The GWAS marker panel was the Illumina OmniExpress Exome microarray (www.illumina.com), consisting of greater than 750,000 SNP markers and greater than 250,000 exonic markers. The group of markers which passed quality checks are referred to as the GWAS Marker Set.

### General Considerations for Data Analysis

The GWAS is hypothesis-free. Markers with unadjusted p<5×10⁻⁸ were considered to be genome-wide significant. In the interests of statistical power, no adjustment was made for multiple endpoints or multiple rounds of analysis.

Table 1 below shows a brief summary of the baseline and demographic characteristics of the 137 patients in PGx-FAS-interim1, the 653 patients in current PGx-FAS-interim2 and the 1669 patients in PGx-FAS-Final. Patients added are more often male, and much less likely to self-report as ‘Oriental’, although a greatly increased percentage now self-report as ‘Asian’; they have lower median baseline ALT.

TABLE 1
Baseline and Demographic Characteristics for PGx-FAS-Interim1, PGx-FAS-Interim2
and PGx-FAS-Final

| | | | PGx-FAS- | | PGx-FAS- | | | |
|---|---|---|---|---|---|---|---|---|
| Variable | Category | Statistics | Interim1 | | Interim2 | | PGx-FAS-Final | |
| Count (n) | | | 137 | | 653 | | 1669 | |
| Sex | Male | n (%) | 88 | (64%) | 433 | (66%) | 1198 | (72%) |
| | Female | n (%) | 49 | (36%) | 220 | (34%) | 471 | (28%) |
| Age (yr) | | Mean (SE) | 32.25 | (0.848) | 38.19 | (0.451) | 39.09 | (0.270) |
| Race | Oriental | n (%) | 119 | (87%) | 270 | (41%) | 464 | (28%) |
| | White | n (%) | 7 | (5%) | 229 | (35%) | 474 | (28%) |
| | Asian | n (%) | 0 | (0%) | 112 | (17%) | 668 | (40%) |
| | Other | n (%) | 11 | (8%) | 42 | (6%) | 63 | (4%) |
| Height (cm) | | Mean (SE) | 168.26 | (0.766) | 167.9 | (0.342) | 168.2 | (0.202) |
| Weight (kg) | | Mean (SE) | 67.74 | (1.43) | 66.93 | (0.597) | 68.9 | (0.358) |
| BMI (kg/m\hat{ }2) | | Mean (SE) | 23.78 | (0.416) | 23.58 | (0.167) | 24.24 | (0.105) |
| Baseline ALT | | Median (IQR) | 123 | (119) | 92 | (104) | 83 | (104) |
| (U/L) | | | | | | | | |

### Quality Checks by Patient

The following criteria were assessed, on the basis of unfiltered GWAS data, in all 1669 patients of any self-reported race (PGx-FAS-Final).
<5% missing genotype data
<30% heterozygosity genome-wide
<30% genotype-concordance with another sample
Reported sex consistent with X-chromosome data
<30% genotype-concordance with another sample

All samples satisfied the criterion of <30% heterozygosity genome-wide. Four samples had 5% or more missing genotypes. Six samples showed X-chromosome homozygosity levels inconsistent with reported sex. All ten were excluded. A further 23 sample-pairs showed high genotype-concordance, consistent with first-degree familial relationship; one of each pair was excluded.

In this way, thirty-three patients were excluded from further analysis; their details are provided in Table 2 below. The remaining 1636 patients, whose genetic data satisfied the criteria above, were incorporated into the PGx-GT-Final Set.

TABLE 2
Thirty-three patients whose genetic data failed quality checks; NA represents
missing

| ANONID | Sample | Protocol | Age | Sex | Race | HBE_BS |
|---|---|---|---|---|---|---|
| 4160 | DNA0007393 | GV28855 | 38 | MALE | ASIAN | POSITIVE |
| 4395 | DNA0006570 | ML21827 | 35 | MALE | ORIENTAL | POSITIVE |
| 4719 | DNA0008408 | GV28855 | 47 | MALE | WHITE | NEGATIVE |
| 4746 | DNA0006560 | GV28855 | 56 | MALE | ASIAN | POSITIVE |
| 4772 | DNA0006340 | ML21827 | 42 | MALE | ORIENTAL | POSITIVE |
| 4861 | DNA0003403 | MV22430 | 51 | FEMALE | ORIENTAL | POSITIVE |
| 5168 | DNA0006298 | ML21827 | 48 | MALE | ORIENTAL | POSITIVE |
| 5337 | DNA0005427 | GV28855 | 32 | MALE | WHITE | POSITIVE |
| 5355 | DNA0005274 | GV28855 | 52 | MALE | WHITE | NEGATIVE |
| 5767 | DNA0006456 | ML21827 | 54 | MALE | ORIENTAL | POSITIVE |
| 5771 | DNA0006558 | GV28855 | 59 | MALE | ASIAN | POSITIVE |
| 5803 | DNA0007574 | GV28855 | 33 | MALE | ASIAN | NEGATIVE |
| 5940 | DNA0008298 | GV28855 | 26 | MALE | ASIAN | POSITIVE |
| 6512 | DNA0005500 | GV28855 | 31 | MALE | ASIAN | POSITIVE |
| 6552 | DNA0008621 | GV28855 | 58 | MALE | ASIAN | NEGATIVE |
| 6818 | DNA0006614 | ML21827 | 61 | MALE | ORIENTAL | POSITIVE |
| 7122 | DNA0005808 | ML18253 | 36 | MALE | WHITE | NEGATIVE |
| 7131 | DNA0006448 | ML21827 | 34 | FEMALE | ORIENTAL | POSITIVE |
| 7470 | DNA0006594 | ML21827 | 57 | FEMALE | ORIENTAL | POSITIVE |
| 7936 | DNA0007494 | GV28855 | 48 | FEMALE | ASIAN | NEGATIVE |
| 7984 | DNA0006220 | ML21827 | 49 | FEMALE | ORIENTAL | POSITIVE |
| 8000 | DNA0003220 | MV22430 | 28 | MALE | ORIENTAL | POSITIVE |
| 8115 | DNA0006322 | ML21827 | 38 | MALE | ORIENTAL | POSITIVE |
| 8150 | DNA0007490 | GV28855 | 31 | FEMALE | ASIAN | POSITIVE |
| 8428 | DNA0007648 | GV28855 | 45 | FEMALE | WHITE | POSITIVE |
| 8618 | DNA0006550 | GV28855 | 29 | MALE | ASIAN | POSITIVE |
| 8623 | DNA0005483 | GV28855 | 61 | MALE | WHITE | POSITIVE |
| 8657 | DNA0006292 | ML21827 | 35 | MALE | ORIENTAL | POSITIVE |
| 8855 | DNA0008452 | GV28855 | 34 | FEMALE | WHITE | NEGATIVE |
| 9654 | DNA0006440 | ML21827 | 52 | MALE | ORIENTAL | POSITIVE |
| 9784 | DNA0007882 | GV28855 | 41 | MALE | ASIAN | POSITIVE |
| 9866 | DNA0003065 | MV22430 | 25 | MALE | WHITE | POSITIVE |
| 9989 | DNA0008453 | GV28855 | 50 | MALE | WHITE | NEGATIVE |

### Quality Checks by Marker

Markers were assessed for missing data. Those with greater than 5% missing data were excluded from further analysis.

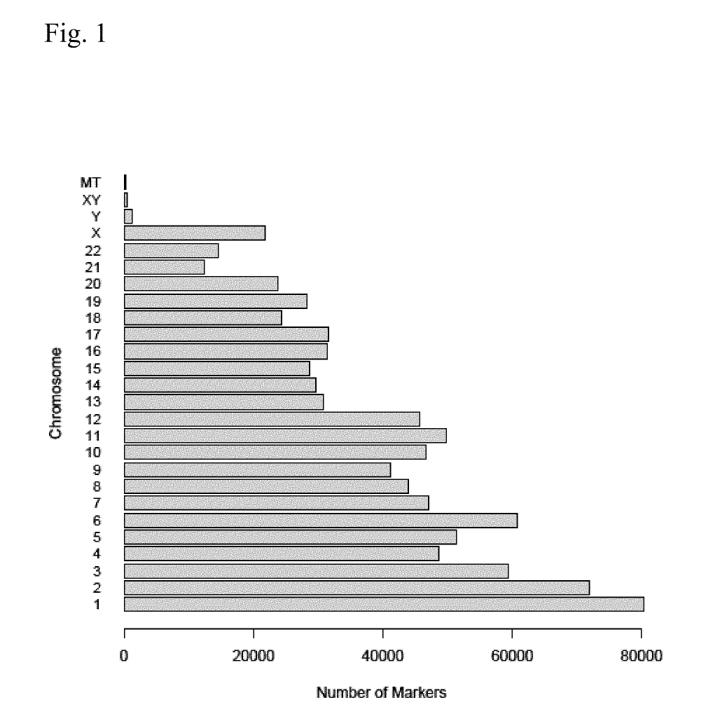

A total of 926,453 markers, with <5% missing overall, were incorporated into the GWAS Marker Set. Their distribution by chromosome is shown in FIG. 1.

### Multivariate Analysis of Ancestry

Principal Components Analysis (PCA) is a technique for reducing the dimensionality of a data set. It linearly transforms a set of variables into a smaller set of uncorrelated variables representing most of the information in the original set (Dunteman, 1989). In the current study, the marker variables were transformed into principal components which were compared to self-reported ethnic groupings. The objective is, in preparation for association testing, to determine clusters of individuals who share a homogeneous genetic background.

A suitable set of 134,575 markers for ancestry analysis was obtained as described in statistical report for Interim Analysis 1. Of this set, 131,974 had at least 5% frequency in the current data. PCA was therefore applied using 131,974 markers, genotyped across 1636 study individuals and 988 HapMap reference individuals (Table 3).

TABLE 3
Details of the HapMap version 3 reference subjects

| Code | Description | Count |
|---|---|---|
| MKK | Maasai in Kinyawa, Kenya | 143 |
| LWK | Luhya in Webuye, Kenya | 90 |
| YRI | Yoruba in Ibadan, Nigeria | 113 |
| ASW | African ancestry in Southwest USA | 49 |
| CEU | Utah residents with Northern and Western European | 112 |
| | ancestry from the CEPH collection | |
| TSI | Tuscans in Italy | 88 |
| MEX | Mexican ancestry in Los Angeles, California | 50 |
| GIH | Gujarati Indians in Houston, Texas | 88 |
| JPT | Japanese in Tokyo, Japan | 86 |
| CHD | Chinese in Metropolitan Denver, Colorado | 85 |
| CHB | Han Chinese in Bejing, China | 84 |
| | TOTAL | 988 |

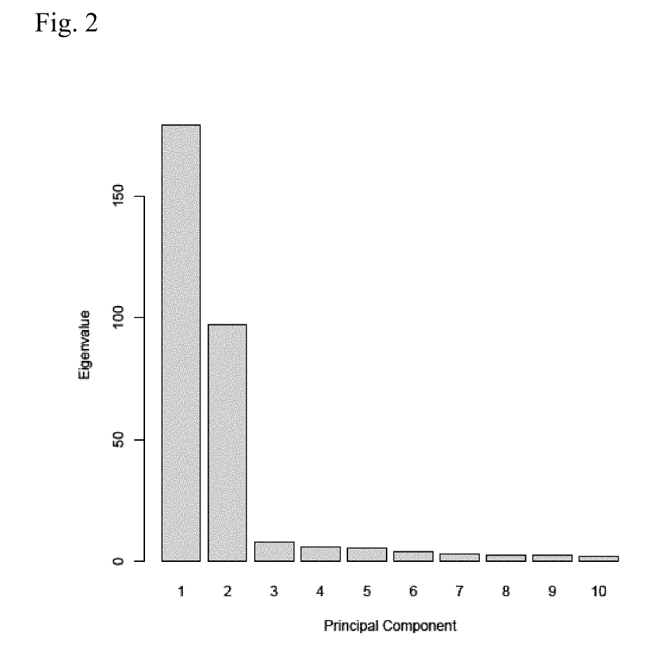

FIG. 2 shows the scree plot of the eigenvalues. It is clear that the majority of information was obtained from the first two principal components of ancestry, with little gain in information from subsequent components.

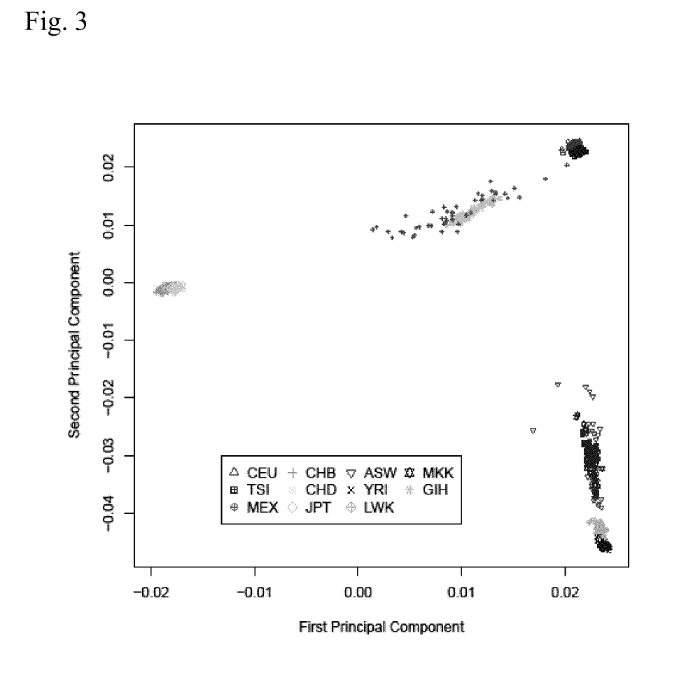

FIG. 3 shows the results of PCA for the HapMap reference data only. Four clusters are visible in this two-dimensional representation. Reading clockwise from top left, they are: Southeast Asian (yellow/blue/green), Mexican (dark green) and South Asian Origin (grey), and Northern and Western European (blue/red) and African origin (blue/orange/ pink/maroon).

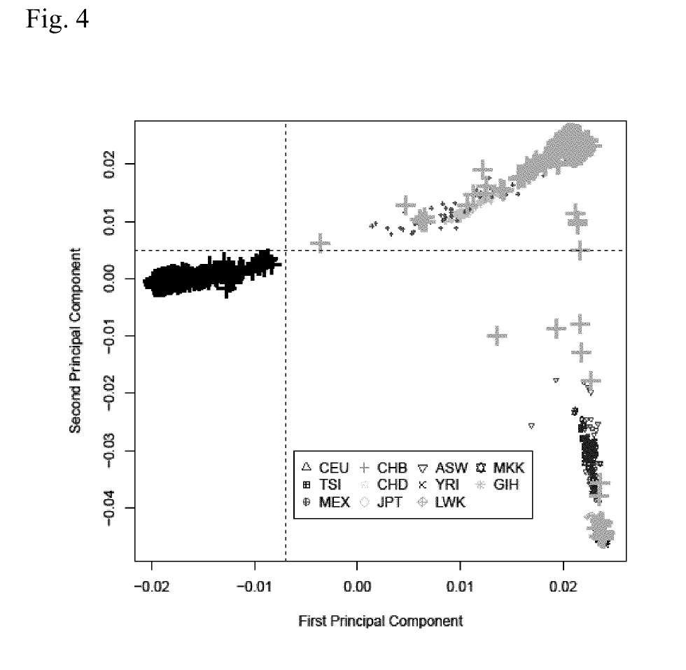

FIG. 4 shows the same data with study participants overlaid as crosses. Patients included in PGx-CN-Final are given by black crosses; patients included in PGx-nonCN-Final are given by grey crosses. As observed in previous interim analyses, the PGx-CN-Final study participants represent a genetically more diverse group of individuals than the reference set. The study participants are likely to have been drawn from different countries in South-East Asia.

For the purposes of genetic analysis, PGx-CN-Final was therefore made up of the 1120 patients falling in a cluster around the Chinese and Japanese reference individuals. A total of 516 patients, whose plotted ancestry clearly departed from that cluster, made up PGx-nonCN-Final.

The number of patients in each analysis is given in Table 4 below. The endpoints are numbered as follows:
1. HBe-positive patients: E-seroconversion or S-loss at >=24-week follow-up
2. HBe-positive patients: (E-seroconversion plus HBV DNA <2000 IU/ml) or S-loss at >=24-week follow-up
3. HBe-negative patients: HBV DNA <2000 IU/ml or S-loss at >=24-week follow-up
4. E-seroconversion or S-loss at >=24-week follow-up if HBe-positive and HBV DNA <2000 IU/ml or S-loss at >=24-week follow-up if HBe-negative (1 and 3)
5. (E-seroconversion plus HBV DNA <2000 IU/ml) or S-loss at >=24-week follow-up if HBe-positive and HBV DNA <2000 IU/ml or S-loss at >=24-week follow-up if HBe-negative (2 and 3)
6. S-loss at >=24-week follow-up

It is noted that 61 patients did not have HBe data, so endpoints 1-5 could not be determined for them. Furthermore, three of the analyses contain at least one group with fewer than 30 patients, and so were not expected to be informative. All analyses were conducted twice: under an additive model of inheritance, and under a dominant mode of inheritance.

### Assessment of Covariates

In order to determine the covariates for the genome-wide association analysis, a series of variables were tested for association with each endpoint, using backwards stepwise regression. In each case, the full model contained the following variables:
Age
Sex
Log(baseline HBV DNA)
Log(ALT)
Genotype
Concomitant nucleotide/nucleoside analogues (NA/Nta)
Study
First principal component of ancestry
Second principal component of ancestry

Backwards steps were taken on the basis of the Akaike Information Criterion (AIC), and covariates were selected separately for each combination of patient set and endpoint. In analysing PGx-GT-Final for endpoint 6, which was un-stratified by HBe status, an indicator variable for inferred Southeast Asian ancestry was imposed. Adjustments for study were applied in all instances.

### Rare and Non-Rare Markers

It is known that allele frequencies vary by ethnic group. In order to perform GWAS analysis for the three key groups of interest, allele frequencies were estimated separately for PGx-CN-Final, PGx-nonCN-Final, and PGx-GT-Final. Due to the differences in sample-sizes, markers were categorised as rare or non-rare, using a frequency threshold of 5% for PGx-nonCN-Final (n=516), 2% for PGx-CN-Final (n=1120), and 1.5% for PGx-GT-Final (n=1636). In this way there were respectively, 605898, 589254 and 651797 non-rare markers available for genome-wide association analysis.

### Univariate Association Analysis

### Methods

Markers were coded in two ways as follows. Firstly they were coded according to an additive model, given by the count of the number of minor alleles. Secondly they were coded according to a dominant model of inheritance, based upon carriage of the minor allele.

Thirty-six rounds of association analysis were conducted due to three patient sets and six endpoints, each under two modes of inheritance. The following model was fitted using multivariate logistic regression:

Endpoint=Intercept+[Covariates]+Marker

Covariates were applied as selected above (Section 8.4).

The significance of each marker was determined using a t-test. The genomic control lambda was calculated for each GWAS analysis and QQ-plots were examined, but no clear evidence of test-statistic inflation was found (Devlin and Roeder 1999). Maximum lambda was 1.05.

All markers were tested, using a chi-square test, for departure from Hardy-Weinberg Equilibrium (HWE) in PGx-GT-Final, PGx-nonCN-Final and PGx-CN-Final.

The results were used to assist in the interpretation of association analysis output. In the tabulated results below, both the minor allele frequency (MAF) and the Hardy-Weinberg result are shown for the relevant, ancestry-defined patient-group.

### Results for Endpoint 1

Covariates were as follows:
PGx-nonCN-HBePos-Final: Log(HBV), Log(ALT), Genotype
PGx-CN-HBePos-Final: Log(HBV), Log(ALT), Genotype , Sex, Study, Concomitant NA/Nta
PGx-GT-HBePos-Final: Log(HBV), Log(ALT), Genotype , Sex, Study, Concomitant NA/Nta

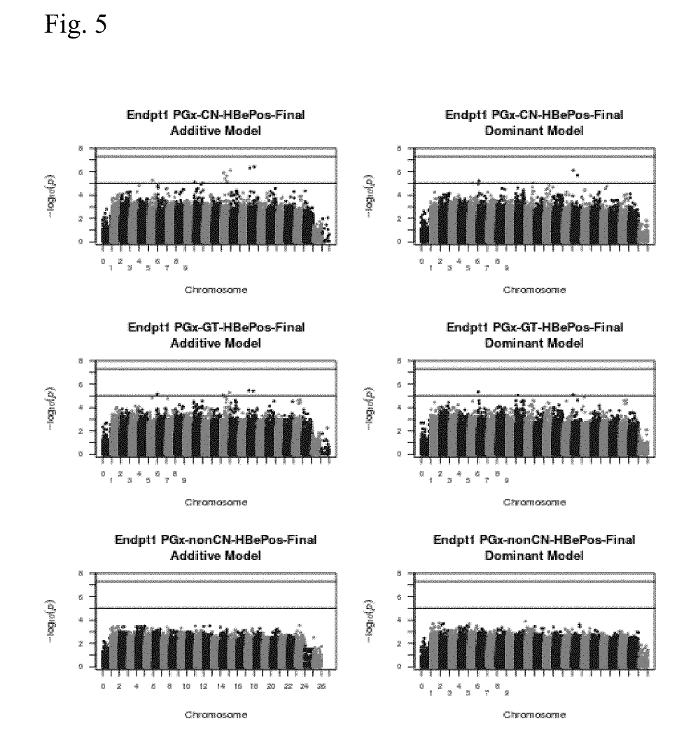

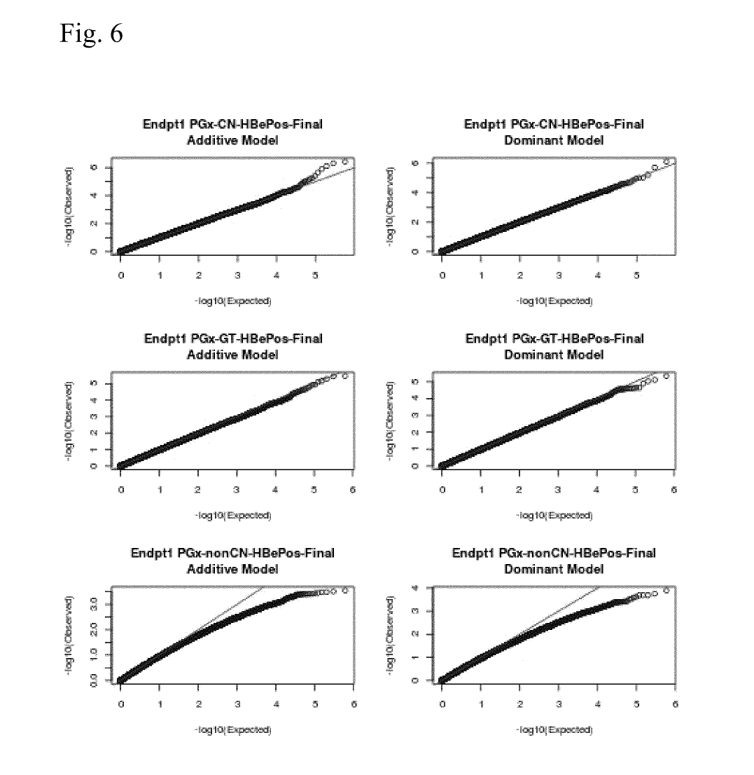

FIGS. 5 and 6 show the Manhattan plots and QQ plots respectively, for Endpoint 1. The first two QQ-lots show deviation above the 45-degree line, indicating the presence of lower p-values than expected by chance alone in PGx-CN-HBePos-Final.

The QQ-plots for PGx-nonCN-HBePos-Final both dip below the 45-degree line, indicating reduced statistical power; the final two Manhattan plots are correspondingly flat. It was noted that there were only 21 responders in these last two analyses.

Details of markers with p<10⁻⁵ are given in Tables 5-8. No marker had p<10⁻⁵ in PGx-nonCN-HBePos-Final, under either mode of inheritance.

TABLE 5
Association Results with p < 10⁻⁵ for Endpoint 1 in PGx-CN-HBePos-Final,
additive model

| Chr | SNP | BP | HWE(p) | MAF | Beta | p-value | Variant | Gene |
|---|---|---|---|---|---|---|---|---|
| 5 | rs1876154 | 10072247 | 0.8929 | 0.1272 | 2.1010 | 5.59e−06 | NA | NA |
| 10 | rs2812338 | 54801847 | 0.7127 | 0.2038 | 1.8580 | 7.66e−06 | NA | NA |
| 10 | rs10824875 | 54828992 | 0.5921 | 0.2125 | 1.8330 | 9.82e−06 | NA | NA |
| 13 | rs1831559 | 111755413 | 0.8839 | 0.2879 | 1.8590 | 1.27e−06 | NA | NA |
| 13 | rs10851257 | 111771610 | 0.3230 | 0.2690 | 1.8340 | 3.96e−06 | NA | NA |
| 13 | rs6492344 | 111748773 | 0.6334 | 0.2509 | 1.8120 | 6.48e−06 | NA | NA |
| 13 | rs12584550 | 111769770 | 1.0000 | 0.3326 | 1.7900 | 2.21e−06 | NA | NA |
| 13 | rs9555773 | 111773108 | 0.6488 | 0.2062 | 1.8610 | 9.02e−06 | NA | NA |
| 13 | rs7983441 | 111749116 | 1.0000 | 0.2902 | 1.8770 | 7.66e−07 | INTERGENIC | NA |
| 16 | rs12446868 | 84593885 | 0.7992 | 0.3776 | 0.5302 | 4.80e−07 | INTERGENIC | NA |
| 16 | rs247878 | 84595354 | 0.8468 | 0.3643 | 0.5209 | 3.70e−07 | DOWNSTREAM | NA |

TABLE 6
Association Results with p < 10⁻⁵ for Endpoint 1 in PGx-CN-HBePos-Final,
dominant model

| Chr | SNP | BP | HWE(p) | MAF | Beta | p-value | Variant | Gene |
|---|---|---|---|---|---|---|---|---|
| 5 | rs1876154 | 10072247 | 0.8929 | 0.1272 | 2.2030 | 9.87e−06 | NA | NA |
| 6 | rs7753766 | 74544376 | 0.1123 | 0.3456 | 2.1200 | 6.05e−06 | INTERGENIC | NA |
| 11 | rs604241 | 133883070 | 0.7965 | 0.3638 | 0.4876 | 9.46e−06 | INTERGENIC | NA |
| 16 | rs12446868 | 84593885 | 0.7992 | 0.3776 | 0.4501 | 7.73e−07 | INTERGENIC | NA |
| 16 | rs247878 | 84595354 | 0.8468 | 0.3643 | 0.4644 | 1.97e−06 | DOWNSTREAM | NA |

TABLE 7
Association Results with p < 10⁻⁵ for Endpoint 1 in PGx-GT-HBePos-Final, additive model

| Chr | SNP | BP | HWE(p) | MAF | Beta | p-value | Variant | Gene |
|---|---|---|---|---|---|---|---|---|
| 6 | rs12210761 | 10176036 | 0.1545 | 0.0462 | 2.7870 | 6.77e−06 | INTERGENIC | NA |
| 13 | rs1831559 | 111755413 | 0.2534 | 0.3506 | 1.7250 | 7.98e−06 | NA | NA |
| 13 | rs7983441 | 111749116 | 0.2331 | 0.3521 | 1.7410 | 5.12e−06 | INTERGENIC | NA |
| 16 | rs12446868 | 84593885 | 0.4569 | 0.3679 | 0.5740 | 3.45e−06 | INTERGENIC | NA |
| 16 | rs247878 | 84595354 | 0.9134 | 0.3493 | 0.5683 | 3.72e−06 | DOWNSTREAM | NA |

TABLE 8
Association Results with p < 10⁻⁵ for Endpoint 1 in PGx-GT-HBePos-Final, dominant model

| Chr | SNP | BP | HWE(p) | MAF | Beta | p-value | Variant | Gene |
|---|---|---|---|---|---|---|---|---|
| 6 | rs12210761 | 10176036 | 0.1545 | 0.0462 | 3.0040 | 4.59e−06 | INTERGENIC | NA |
| 10 | rs1411283 | 27302772 | 0.0422 | 0.4801 | 0.4873 | 9.25e−06 | INTRONIC | ANKRD26 |
| 16 | rs12446868 | 84593885 | 0.4569 | 0.3679 | 0.5002 | 7.72e−06 | INTERGENIC | NA |

### Results for Endpoint 2

Covariates were as follows:
PGx-nonCN-HBePos-Final: Log(HBV), Log(ALT), Genotype
PGx-CN-HBePos-Final: Log(HBV), Log(ALT), Genotype, Age, Study, Concomitant NA/Nta
PGx-GT-HBePos-Final: Log(HBV), Log(ALT), Genotype, Age, Study, Concomitant NA/Nta

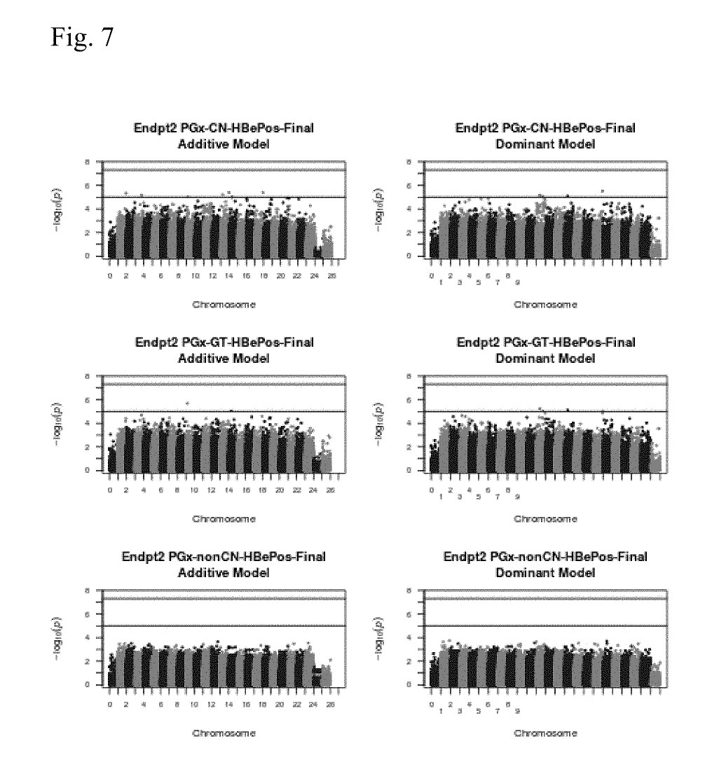

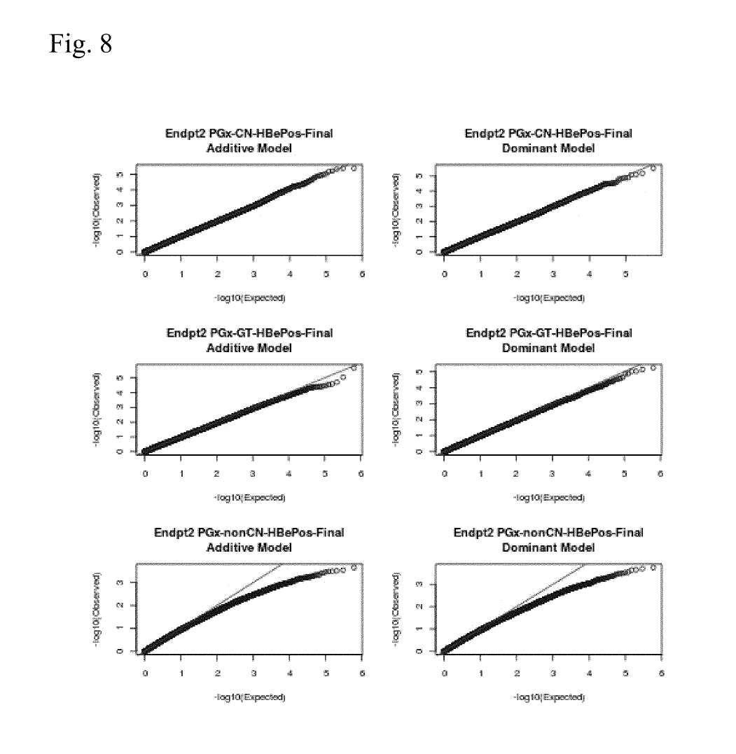

FIGS. 7 and 8 show the Manhattan Plots and QQ plots respectively, for Endpoint 2. Details of markers with p<10⁻⁵ are given in Tables 9-12. No marker had p<10⁻⁵ in PGx-nonCN-HBeNeg-Final, under either mode of inheritance. It was noted that there were only 18 responders: The QQ-plots were seen to curve downwards and the Manhattan plots were depressed.

TABLE 9
Association Results with p < 10⁻⁵ for Endpoint 2 in PGx-CN-HBePos-Final, additive model

| Chr | SNP | BP | HWE(p) | MAF | Beta | p-value | Variant | Gene |
|---|---|---|---|---|---|---|---|---|
| 1 | rs11163805 | 84168682 | 0.6108 | 0.2888 | 1.8610 | 4.70e−06 | INTERGENIC | NA |
| 3 | rs6443144 | 7983344 | 0.8685 | 0.2364 | 1.9390 | 6.74e−06 | INTERGENIC | NA |
| 9 | rs11139349 | 84244131 | 0.0953 | 0.2703 | 1.8360 | 9.52e−06 | INTRONIC | TLE1 |
| 13 | rs1831559 | 111755413 | 0.8839 | 0.2879 | 1.9060 | 6.08e−06 | NA | NA |
| 13 | rs7983441 | 111749116 | 1.0000 | 0.2902 | 1.9240 | 4.04e−06 | INTERGENIC | NA |
| 17 | rs11868362 | 55498236 | 0.4205 | 0.1536 | 0.3363 | 4.07e−06 | INTRONIC | MSI2 |

TABLE 10
Association Results with p < 10⁻⁵ for Endpoint 2 in PGx-CN-HBePos-Final, dominant model

| Chr | SNP | BP | HWE(p) | MAF | Beta | p-value | Variant | Gene |
|---|---|---|---|---|---|---|---|---|
| 11 | rs1384010 | 107595881 | 0.7118 | 0.4129 | 2.6700 | 6.66e−06 | DOWNSTREAM | NA |
| 11 | rs1351518 | 107614258 | 0.0075 | 0.2929 | 2.3000 | 8.44e−06 | INTERGENIC | NA |
| 14 | rs1157322 | 79074088 | 0.7694 | 0.0549 | 3.0120 | 7.94e−06 | NA | NA |
| 17 | rs11868362 | 55498236 | 0.4205 | 0.1536 | 0.3108 | 3.10e−06 | INTRONIC | MSI2 |

TABLE 11
Association Results with p < 10⁻⁵ for Endpoint 2 in PGx-GT-HBePos-Final, additive model

| Chr | SNP | BP | HWE(p) | MAF | Beta | p-value | Variant | Gene |
|---|---|---|---|---|---|---|---|---|
| 9 | rs11139349 | 84244131 | 0.1680 | 0.2706 | 1.8610 | 2.07e−06 | INTRONIC | TLE1 |
| 14 | rs1157322 | 79074088 | 1.0000 | 0.0394 | 2.8290 | 8.99e−06 | NA | NA |

TABLE 12
Association Results with p < 10⁻⁵ for Endpoint 2 in PGx-GT-HBePos-Final, dominant model

| Chr | SNP | BP | HWE(p) | MAF | Beta | p-value | Variant | Gene |
|---|---|---|---|---|---|---|---|---|
| 11 | rs1384010 | 107595881 | 0.0056 | 0.4994 | 2.6260 | 5.73e−06 | DOWNSTREAM | NA |
| 11 | rs1351518 | 107614258 | 2.24e−06 | 0.3796 | 2.2220 | 9.46e−06 | INTERGENIC | NA |
| 14 | rs1157322 | 79074088 | 1.0000 | 0.0394 | 2.9500 | 7.31e−06 | NA | NA |
| 17 | rs646097 | 37076331 | 0.1971 | 0.3590 | 0.4639 | 9.45e−06 | 3PRIME_UTR | LASP1 |

### Results for Endpoint 3

Covariates were as follows:
PGx-nonCN-HBeNeg-Final : Log(HBV)
PGx-CN-HBeNeg-Final: Log(HBV), Log(ALT), Genotype, 2nd PC, Study
PGx-GT-HBeNeg-Final: Log(HBV), Genotype, PC1, PC2

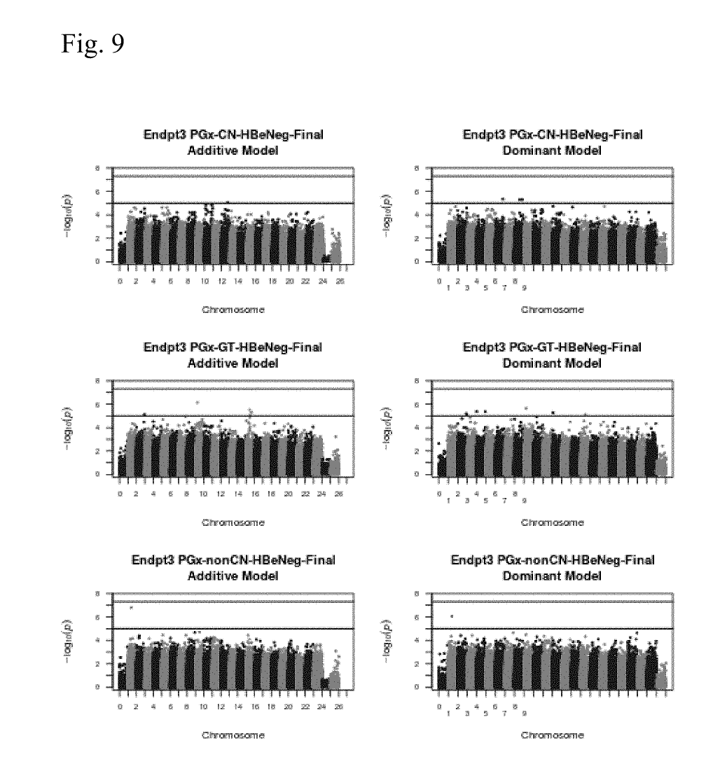

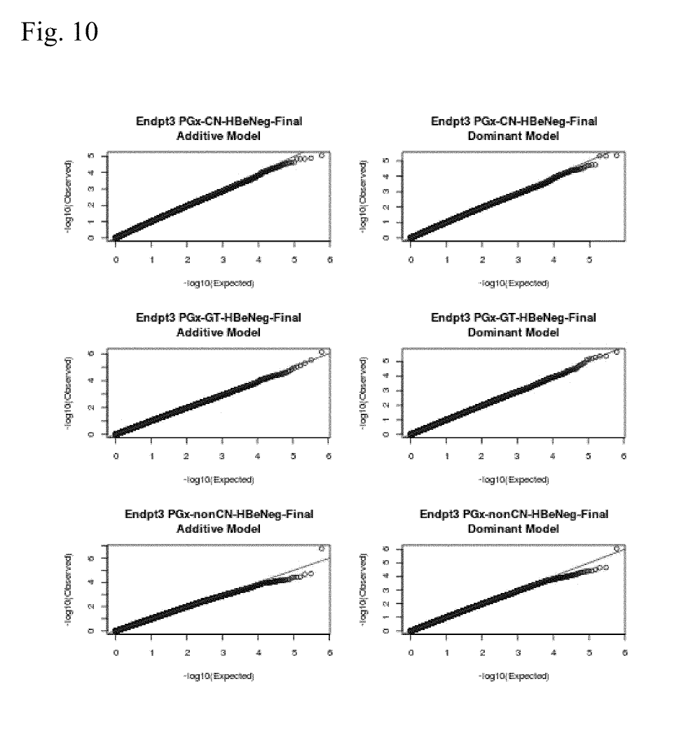

FIGS. 9 and 10 show the Manhattan Plots and QQ plots respectively, for Endpoint 3. Details of markers with p<10⁻⁵ are given in Tables 13-18. It was noted that despite some evidence of reduced statistical power, a single marker on chromosome 1 had p<10-6 for both modes of inheritance in PGx-nonCN-HBeNeg-Final.

TABLE 13
Association Results with p < 10⁻⁵ for Endpoint 3 in PGx-nonCN-HBeNeg-Final, additive model

| Chr | SNP | BP | HWE(p) | MAF | Beta | p-value | Variant | Gene |
|---|---|---|---|---|---|---|---|---|
| 1 | rs17037122 | 11689663 | 0.3718 | 0.1434 | 4.3170 | 1.58e−07 | INTERGENIC | NA |

TABLE 14
Association Results with p < 10⁻⁵ for Endpoint 3 in PGx-nonCN-HBeNeg-Final, dominant model

| Chr | SNP | BP | HWE(p) | MAF | Beta | p-value | Variant | Gene |
|---|---|---|---|---|---|---|---|---|
| 1 | rs17037122 | 11689663 | 0.3718 | 0.1434 | 4.2450 | 8.77e−07 | INTERGENIC | NA |

TABLE 15
Association Results with p < 10⁻⁵ for Endpoint 3 in PGx-CN-HBeNeg-Final, additive model

| Chr | SNP | BP | HWE(p) | MAF | Beta | p-value | Variant | Gene |
|---|---|---|---|---|---|---|---|---|
| 12 | rs2464266 | 115840082 | 0.3273 | 0.1874 | 0.2583 | 8.79e−06 | INTERGENIC | NA |

TABLE 16
Association Results with p < 10⁻⁵ for Endpoint 3 in PGx-CN-HBeNeg-Final, dominant model

| Chr | SNP | BP | HWE(p) | MAF | Beta | p-value | Variant | Gene |
|---|---|---|---|---|---|---|---|---|
| 6 | rs9496139 | 142093922 | 0.6191 | 0.2369 | 0.1812 | 4.52e−06 | INTERGENIC | NA |
| 8 | rs2014238 | 76299353 | 0.2058 | 0.3072 | 5.8110 | 4.97e−06 | INTERGENIC | NA |
| 8 | rs2980231 | 76296789 | 0.2625 | 0.3080 | 5.8110 | 4.97e−06 | INTERGENIC | NA |

TABLE 17
Association Results with p < 10⁻⁵ for Endpoint 3 in PGx-GT-HBeNeg-Final, additive model

| Chr | SNP | BP | HWE(p) | MAF | Beta | p-value | Variant | Gene |
|---|---|---|---|---|---|---|---|---|
| NA | exm2237722 | NA | 0.0358 | 0.0339 | 0.1070 | 7.48e−06 | NA | NA |
| 9 | rs16924016 | 100511331 | 0.9242 | 0.1541 | 0.3357 | 7.25e−07 | INTERGENIC | NA |
| 15 | rs2899723 | 67736023 | 0.4542 | 0.3631 | 2.0360 | 2.89e−06 | INTRONIC | IQCH |
| 15 | rs8027115 | 67819115 | 0.5936 | 0.3651 | 1.9480 | 9.12e−06 | DOWNSTREAM | NA |
| NA | exm2267780 | NA | 0.5195 | 0.3597 | 2.0100 | 4.94e−06 | NA | NA |

TABLE 18
Association Results with p < 10⁻⁵ for Endpoint 3 in PGx-GT-HBeNeg-Final, dominant model

| Chr | SNP | BP | HWE(p) | MAF | Beta | p-value | Variant | Gene |
|---|---|---|---|---|---|---|---|---|
| 2 | rs9973954 | 19885907 | 2.04e−39 | 0.2803 | 0.2975 | 6.27e−06 | INTERGENIC | NA |
| NA | exm2237722 | NA | 0.0358 | 0.0339 | 0.1009 | 6.96e−06 | NA | NA |
| 4 | rs1040084 | 54410224 | 0.5610 | 0.3069 | 2.3660 | 4.26e−06 | INTRONIC | LNX1 |
| 4 | rs1913484 | 54410324 | 0.3796 | 0.3423 | 2.3610 | 4.35e−06 | INTRONIC | LNX1 |
| 9 | rs16924016 | 100511331 | 0.9242 | 0.1541 | 0.3186 | 2.21e−06 | INTERGENIC | NA |
| NA | exm1010813 | NA | 0.0224 | 0.0535 | 0.1299 | 5.23e−06 | NA | NA |
| 15 | rs6576456 | 26009240 | 0.0050 | 0.2725 | 0.4112 | 7.57e−06 | INTRONIC | ATP10A |

### Results for Endpoint 4

Covariates were as follows:
PGx-nonCN-Final : Log(HBV), Genotype
PGx-CN-Final: Log(HBV), Genotype, Log(ALT), Study, Concomitant NA/Nta
PGx-GT-Final: Log(HBV), Genotype, Log(ALT), Study, Concomitant NA/Nta, PC1

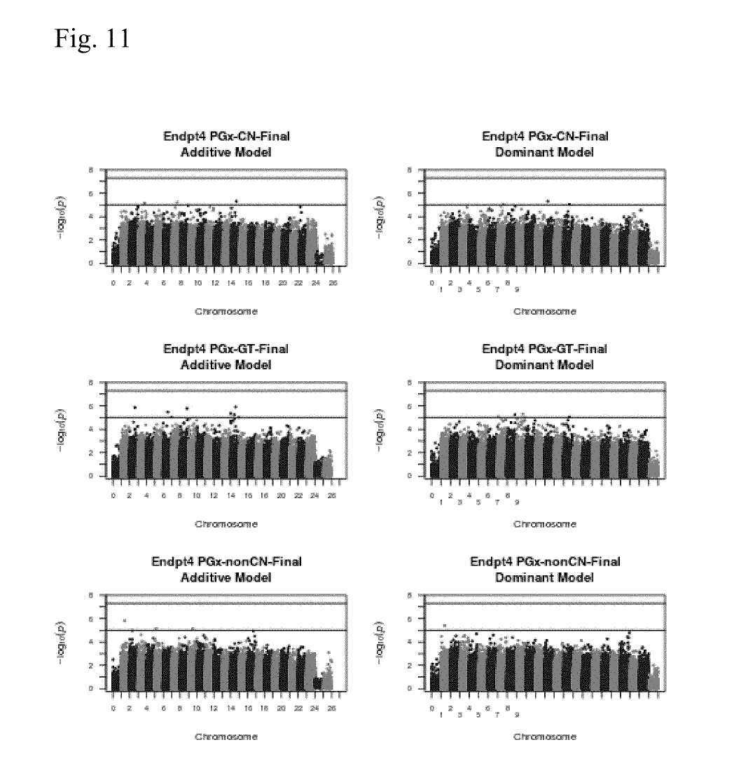

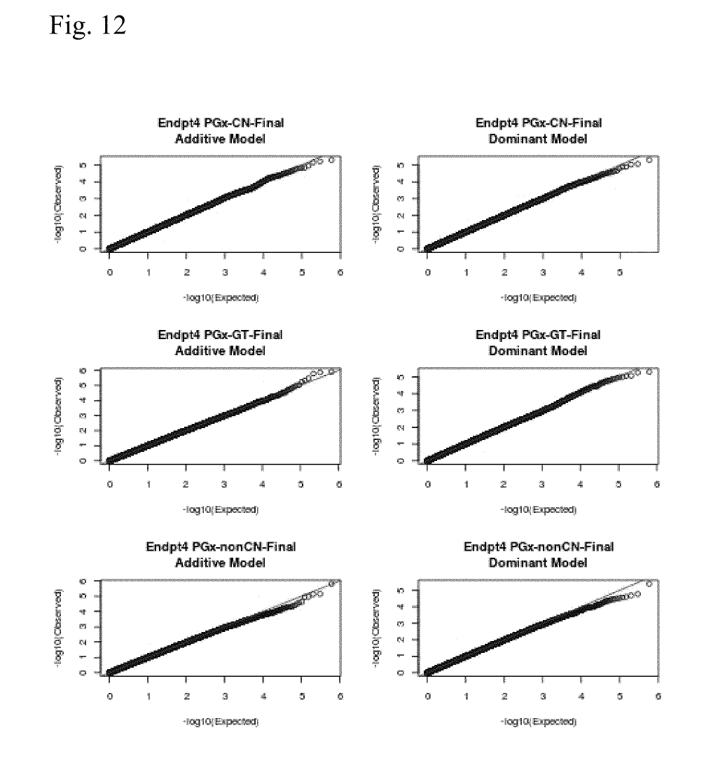

FIGS. 11 and 12 show the Manhattan Plots and QQ plots respectively, for Endpoint 4. Details of markers with p<10⁻⁵ are given in Tables 19-24.

TABLE 19
Association Results with p < 10⁻⁵ for Endpoint 4 in PGx-nonCN-Final, additive model

| Chr | SNP | BP | HWE(p) | MAF | Beta | p-value | Variant | Gene |
|---|---|---|---|---|---|---|---|---|
| 1 | rs17037122 | 11689663 | 0.3718 | 0.1434 | 2.9930 | 1.53e−06 | INTERGENIC | NA |
| 5 | rs10475403 | 8062594 | 1.0000 | 0.4398 | 0.5024 | 7.08e−06 | INTERGENIC | NA |
| 9 | rs715243 | 87216845 | 0.3317 | 0.4709 | 0.5078 | 7.27e−06 | DOWNSTREAM | NA |

TABLE 20
Association Results with p < 10⁻⁵ for Endpoint 4 in PGx-nonCN-Final, dominant model

| Chr | SNP | BP | HWE(p) | MAF | Beta | p-value | Variant | Gene |
|---|---|---|---|---|---|---|---|---|
| 1 | rs17037122 | 11689663 | 0.3718 | 0.1434 | 3.1610 | 3.97e−06 | INTERGENIC | NA |

TABLE 21
Association Results with p < 10⁻⁵ for Endpoint 4 in PGx-CN-Final, additive model

| Chr | SNP | BP | HWE(p) | MAF | Beta | p-value | Variant | Gene |
|---|---|---|---|---|---|---|---|---|
| 3 | rs6443144 | 7983344 | 0.8685 | 0.2364 | 1.6780 | 6.86e−06 | INTERGENIC | NA |
| 7 | rs2189452 | 120462955 | 0.4714 | 0.2933 | 0.6008 | 5.96e−06 | NA | NA |
| 14 | rs9324018 | 100781877 | 0.5271 | 0.2531 | 1.6680 | 4.89e−06 | INTERGENIC | NA |

TABLE 22
Association Results with p < 10⁻⁵ for Endpoint 4 in PGx-CN-Final, dominant model

| Chr | SNP | BP | HWE(p) | MAF | Beta | p-value | Variant | Gene |
|---|---|---|---|---|---|---|---|---|
| 7 | rs2189452 | 120462955 | 0.4714 | 0.2933 | 0.5360 | 8.34e−06 | NA | NA |
| 12 | rs7968170 | 16149335 | 0.5504 | 0.4982 | 0.4869 | 4.87e−06 | INTRONIC | DERA |
| 14 | rs9324018 | 100781877 | 0.5271 | 0.2531 | 1.8700 | 9.18e−06 | INTERGENIC | NA |

TABLE 23
Association Results with p < 10⁻⁵ for Endpoint 4 in PGx-GT-Final, additive model

| Chr | SNP | BP | HWE(p) | MAF | Beta | p-value | Variant | Gene |
|---|---|---|---|---|---|---|---|---|
| 2 | rs9287655 | 15385484 | 0.5813 | 0.4419 | 0.6617 | 1.37e−06 | INTRONIC | ENSG151779 |
| 6 | rs2803073 | 162962828 | 3.0e−20 | 0.4232 | 1.5160 | 3.39e−06 | INTRONIC | PARK2 |
| 6 | rs1937590 | 154036895 | 0.3073 | 0.1247 | 1.7220 | 9.27e−06 | INTERGENIC | NA |
| 8 | rs2945861 | 8283667 | 0.0357 | 0.1901 | 0.5957 | 1.66e−06 | INTERGENIC | NA |
| 14 | rs1997894 | 85977518 | 0.7228 | 0.4277 | 0.6814 | 4.55e−06 | INTERGENIC | NA |
| 14 | rs1495471 | 57920445 | 0.7865 | 0.2404 | 1.5540 | 5.68e−06 | INTERGENIC | NA |
| 14 | rs9324018 | 100781877 | 0.0334 | 0.2983 | 1.5400 | 1.25e−06 | INTERGENIC | NA |
| 14 | rs1152537 | 57931444 | 2.4e−05 | 0.1219 | 1.7800 | 9.82e−06 | INTERGENIC | NA |

TABLE 24
Association Results with p < 10⁻⁵ for Endpoint 4 in PGx-GT-Final, dominant model

| Chr | SNP | BP | HWE(p) | MAF | Beta | p-value | Variant | Gene |
|---|---|---|---|---|---|---|---|---|
| 7 | rs10236906 | 18739670 | 0.1231 | 0.1748 | 0.5664 | 8.46e−06 | INTRONIC | HDAC9 |
| 8 | rs2945861 | 8283667 | 0.0357 | 0.1901 | 0.5576 | 5.62e−06 | INTERGENIC | NA |
| 9 | rs7042473 | 99346570 | 0.2472 | 0.2598 | 1.7050 | 4.97e−06 | INTRONIC | CDC14B, |
| | | | | | | | | CDC14C |
| 9 | rs2077415 | 1742374 | 0.8024 | 0.4435 | 0.5732 | 9.97e−06 | INTERGENIC | NA |
| 14 | rs9324018 | 100781877 | 0.0334 | 0.2983 | 1.6850 | 8.64e−06 | INTERGENIC | NA |

### Results for Endpoint 5

Covariates were as follows:
PGx-nonCN-Final: Log(HBV), Genotype
PGx-CN-Final: Log(HBV), Genotype, Log(ALT), Study, Concomitant NA/Nta
PGx-GT-Final: Log(HBV), Genotype, Log(ALT), Study, Concomitant NA/Nta, PC1

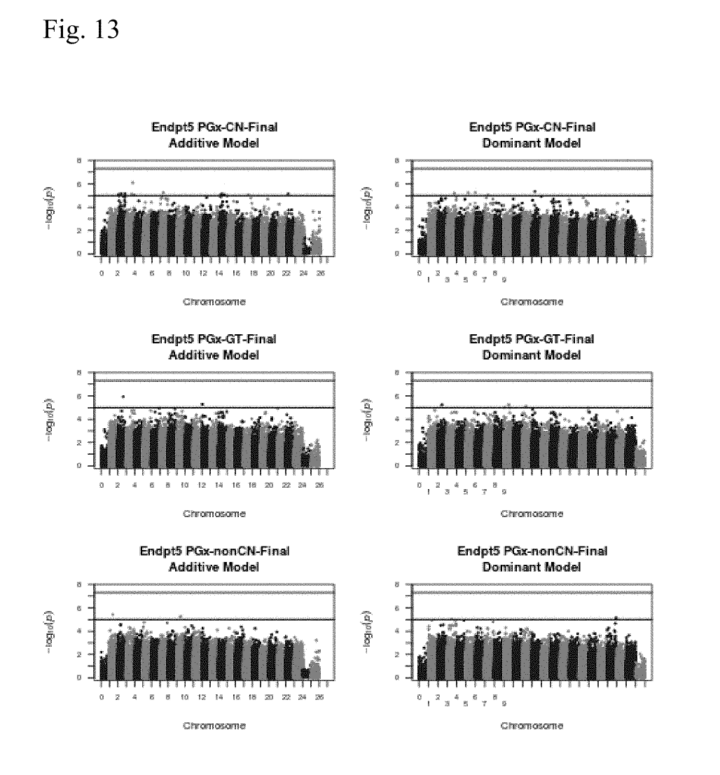

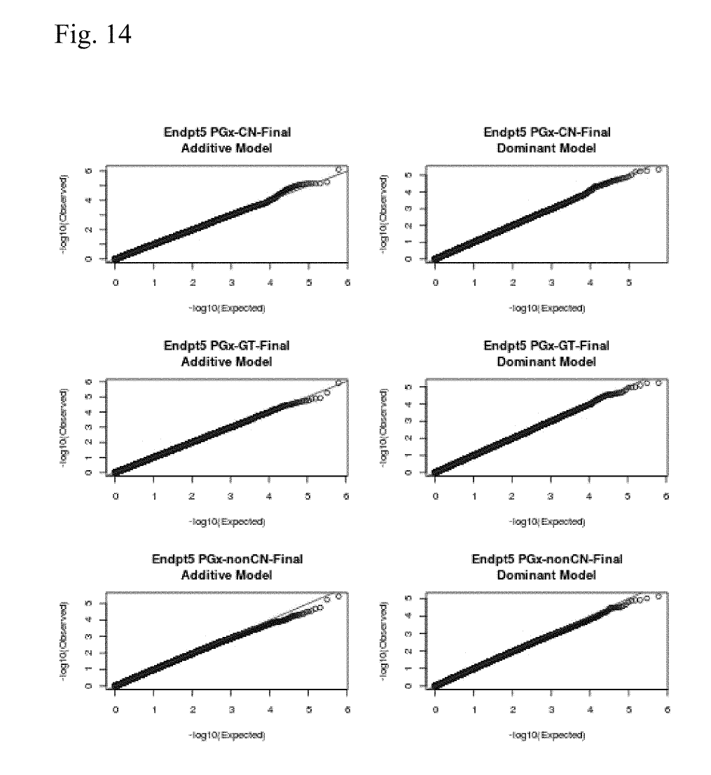

FIGS. 13 and 14 show the Manhattan Plots and QQ plots respectively, for Endpoint 5. Details of markers with p<10⁻⁵ are given in Tables 25-30. Under an additive model, a suggestive association (p=8.05e-06) with a non-synonymous change in CENPO (Centromere Protein 0) was observed in PGx-CN-Final.

TABLE 25
Association Results with p < 10⁻⁵ for Endpoint 5 in PGx-nonCN-Final, additive model

| Chr | SNP | BP | HWE(p) | MAF | Beta | p-value | Variant | Gene |
|---|---|---|---|---|---|---|---|---|
| 1 | rs17037122 | 11689663 | 0.3718 | 0.1434 | 2.8740 | 3.68e−06 | INTERGENIC | NA |
| 9 | rs715243 | 87216845 | 0.3317 | 0.4709 | 0.5000 | 5.77e−06 | DOWNSTREAM | NA |

TABLE 26
Association Results with p < 10⁻⁵ for Endpoint 5 in PGx-nonCN-Final, dominant model

| Chr | SNP | BP | HWE(p) | MAF | Beta | p-value | Variant | Gene |
|---|---|---|---|---|---|---|---|---|
| 3 | rs2302503 | 37107470 | 0.0568 | 0.4157 | 0.3719 | 9.50e−06 | INTRONIC | LRRFIP2 |
| 20 | rs6015181 | 56647166 | 0.7677 | 0.3343 | 2.6740 | 7.41e−06 | INTERGENIC | NA |

TABLE 27
Association Results with p < 10⁻⁵ for Endpoint 5 in PGx-CN-Final, additive model

| Chr | SNP | BP | HWE(p) | MAF | Beta | p-value | Variant | Gene |
|---|---|---|---|---|---|---|---|---|
| 2 | rs1550116 | 25022598 | 0.6000 | 0.2195 | 0.5565 | 8.05e−06 | NON- | CENPO |
| | | | | | | | SYNONYMOUS | |
| 2 | rs1550115 | 25041620 | 0.9315 | 0.2241 | 0.5565 | 7.02e−06 | INTRONIC | CENPO |
| 2 | rs2082881 | 25038268 | 0.7969 | 0.2246 | 0.5565 | 7.02e−06 | INTRONIC | CENPO |
| NA | exm2265462 | NA | 0.8799 | 0.2724 | 0.5730 | 7.43e−06 | NA | NA |
| 3 | rs6443144 | 7983344 | 0.8685 | 0.2364 | 1.8390 | 7.76e−07 | INTERGENIC | NA |
| 3 | rs1403069 | 70274229 | 0.8206 | 0.2715 | 0.5769 | 8.94e−06 | INTERGENIC | NA |
| 7 | rs9691873 | 28730009 | 0.6013 | 0.0938 | 2.2250 | 5.71e−06 | INTRONIC | CREB5 |
| 14 | rs8012912 | 70474207 | 0.9016 | 0.4080 | 1.6200 | 7.29e−06 | INTRONIC | SMOC1 |
| 14 | rs11158827 | 70479174 | 0.5797 | 0.4152 | 1.6200 | 8.28e−06 | INTRONIC | SMOC1 |
| 17 | rs11870323 | 52928826 | 0.0421 | 0.0982 | 2.1120 | 8.85e−06 | INTERGENIC | NA |
| 22 | rs4821558 | 37308785 | 0.1054 | 0.3987 | 0.6146 | 7.17e−06 | INTERGENIC | NA |

TABLE 28
Association Results with p < 10⁻⁵ for Endpoint 5 in PGx-CN-Final, dominant model

| Chr | SNP | BP | HWE(p) | MAF | Beta | p-value | Variant | Gene |
|---|---|---|---|---|---|---|---|---|
| 3 | rs6443144 | 7983344 | 0.8685 | 0.2364 | 1.9800 | 6.29e−06 | INTERGENIC | NA |
| 5 | rs1692421 | 71319752 | 0.4086 | 0.2732 | 0.5036 | 5.79e−06 | INTERGENIC | NA |
| 5 | rs1692423 | 71319262 | 0.4523 | 0.2737 | 0.5029 | 5.53e−06 | INTERGENIC | NA |
| 7 | rs9691873 | 28730009 | 0.6013 | 0.0938 | 2.3190 | 9.46e−06 | INTRONIC | CREB5 |
| 12 | rs7968170 | 16149335 | 0.5504 | 0.4982 | 0.4634 | 4.50e−06 | INTRONIC | DERA |

TABLE 29
Association Results with p < 10⁻⁵ for Endpoint 5 in PGx-GT-Final, additive model

| Chr | SNP | BP | HWE(p) | MAF | Beta | p-value | Variant | Gene |
|---|---|---|---|---|---|---|---|---|
| 2 | rs9287655 | 15385484 | 0.5813 | 0.4419 | 0.6444 | 1.18e−06 | INTRONIC | ENSG00000151779 |
| 12 | rs216312 | 6128984 | 0.3358 | 0.3625 | 0.6621 | 5.31e−06 | INTRONIC | VWF |

TABLE 30
Association Results with p < 10⁻⁵ for Endpoint 5 in PGx-GT-Final, dominant model

| Chr | SNP | BP | HWE(p) | MAF | Beta | p-value | Variant | Gene |
|---|---|---|---|---|---|---|---|---|
| 2 | rs993147 | 185209989 | 0.0028 | 0.1886 | 0.5210 | 5.93e−06 | INTERGENIC | NA |
| 9 | rs10978436 | 99400209 | 0.5423 | 0.2404 | 1.7610 | 9.97e−06 | DOWNSTREAM | NA |
| 9 | rs2370220 | 917667 | 0.3624 | 0.1427 | 0.5233 | 5.81e−06 | INTRONIC | DMRT1 |
| 11 | rs2279519 | 123477352 | 0.8812 | 0.2090 | 1.7460 | 8.02e−06 | SYNONYMOUS | GRAMD1B |
| | | | | | | | CODING | |

### Results for Endpoint 6

Covariates were as follows:
PGx-nonCN-Final: Log(ALT), Genotype
PGx-CN-Final: Log(HBV), Genotype, Concomitant NA/Nta, PC1
PGx-GT-Final: Log(ALT), Genotype, Concomitant NA/Nta, CN

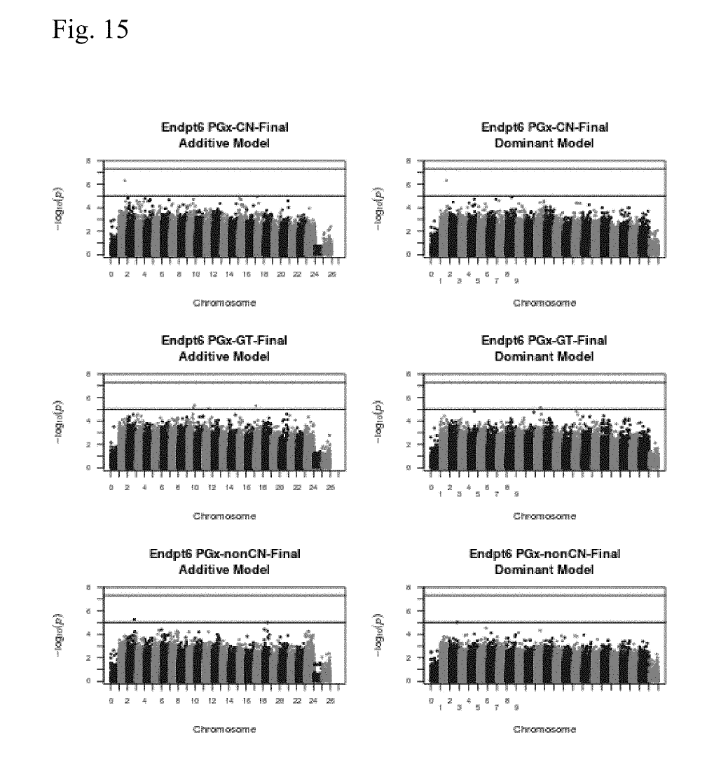

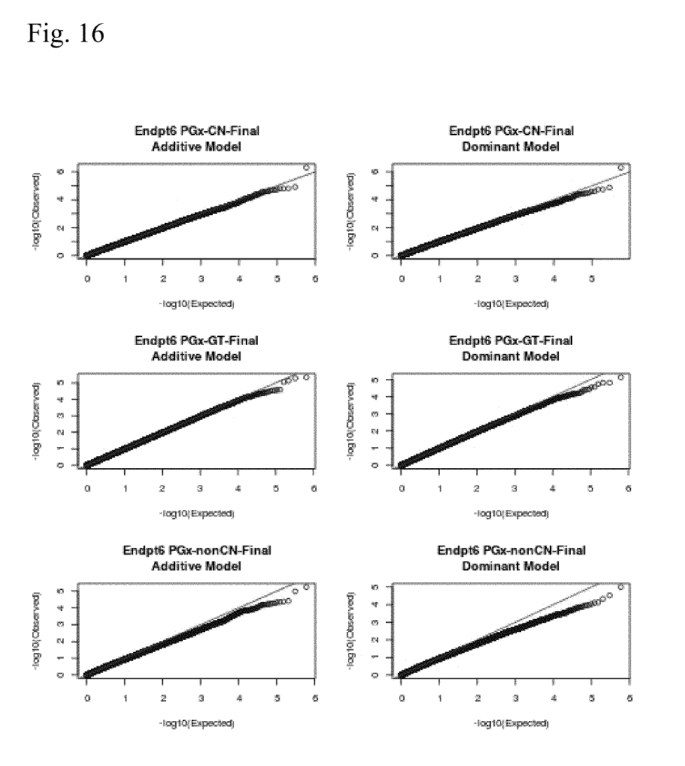

FIGS. 15 and 16 show the Manhattan Plots and QQ plots respectively, for Endpoint 6. Details of markers with p<10⁻⁵ are given in Tables 31-36. A single marker on chromosome 1, in the 3′UTR of FCER1A (Fc Fragment of IgE, High Affinity I Receptor For Alpha Polypeptide) had p<10⁻⁶ and dominated results for PGx-CN-Final.

TABLE 31
Association Results with p < 10⁻⁵ for Endpoint 6 in PGx-nonCN-Final, additive model

| Chr | SNP | BP | HWE(p) | MAF | Beta | p-value | Variant | Gene |
|---|---|---|---|---|---|---|---|---|
| 2 | rs12992677 | 232400839 | 0.1633 | 0.1473 | 5.7670 | 5.58e−06 | INTERGENIC | NA |

TABLE 32
Association Results with p < 10⁻⁵ for Endpoint 6 in PGx-nonCN-Final, dominant model

| Chr | SNP | BP | HWE(p) | MAF | Beta | p-value | Variant | Gene |
|---|---|---|---|---|---|---|---|---|
| 2 | rs12992677 | 232400839 | 0.1633 | 0.1473 | 8.6340 | 9.90e−06 | INTERGENIC | NA |

TABLE 33
Association Results with p < 10⁻⁵ for Endpoint 6 in PGx-CN-Final, additive model

| Chi | SNP | BP | HWE(p) | MAF | Beta | p-value | Variant | Gene |
|---|---|---|---|---|---|---|---|---|
| 1 | rs7549785 | 159277868 | 1.0000 | 0.0201 | 8.2240 | 4.83e−07 | 3PRIME_UTR | FCER1A |

TABLE 34
Association Results with p < 10⁻⁵ for Endpoint 6 in PGx-CN-Final, dominant model

| Chi | SNP | BP | HWE(p) | MAF | Beta | p-value | Variant | Gene |
|---|---|---|---|---|---|---|---|---|
| 1 | rs7549785 | 159277868 | 1.0000 | 0.0201 | 8.2240 | 4.83e−07 | 3PRIME_UTR | FCER1A |

TABLE 35
Association Results with p < 10⁻⁵ for Endpoint 6 in PGx-GT-Final, additive model

| Chr | SNP | BP | HWE(p) | MAF | Beta | p-value | Variant | Gene |
|---|---|---|---|---|---|---|---|---|
| 9 | rs10814834 | 4086370 | 0.0055 | 0.3817 | 0.4571 | 7.38e−06 | INTRONIC | GLIS3 |
| 9 | rs10491723 | 100927632 | 0.0911 | 0.3745 | 2.1090 | 4.51e−06 | INTRONIC | CORO2A |
| 11 | rs6592052 | 82268478 | 0.1522 | 0.0208 | 6.6180 | 8.78e−06 | INTERGENIC | NA |
| 17 | rs16943470 | 57446588 | 0.0208 | 0.0645 | 2.9650 | 5.21e−06 | INTRONIC | YPEL2 |

TABLE 36
Association Results with p < 10⁻⁵ for Endpoint 6 in PGx-GT-Final, dominant model

| Chr | SNP | BP | HWE(p) | MAF | Beta | p-value | Variant | Gene |
|---|---|---|---|---|---|---|---|---|
| 11 | rs6592052 | 82268478 | 0.1522 | 0.0208 | 7.8720 | 7.20e−06 | INTERGENIC | NA |

### Interpretation

No marker achieved genome-wide significance (p<5×10⁻⁸) in association analysis with any endpoint however, ten associations surpassed p<1×10⁻⁶.

The majority of suggestive associations lay in intergenic regions however 27 markers mapped within the boundaries of 24 genes. They are listed in Table 37 below.

Some of the -highlighted genes have been implicated, either directly or indirectly in the mechanism of hepatitis B-associated hepatocellular carcinoma. For example, Von Willebrand Factor (VFW) is a published biomarker of tumour development in hepatitis B virus-associated human hepatocellular carcinoma (Liu et al, 2014). Also, hepatitis B virus X protein has been shown to play a role in the regulation of LASP1 expression, to mediate proliferation and migration of hepatoma cells (Tang et al, 2012). The single non-synonymous change tabulated lies in CENPO. It has been noted that Hepatitis B virus X protein mutant up-regulates CENP-A expression in hepatoma cells (Liu et al, 2012).

TABLE 37
Gene-based markers associated with one or more endpoint in the current
analysis

| SNP | Chr | HG18 | GeneVariant | GeneName | GeneDescription |
|---|---|---|---|---|---|
| rs7549785 | 1 | 157544492 | 3PRIME UTR | FCER1A | High affinity immunoglobulin |
| | | | | | epsilon receptor subunit alpha |
| | | | | | precursor (FcERI) (IgE Fc |
| | | | | | receptor subunit alpha) (Fc- |
| | | | | | epsilon RI-alpha) |
| rs9287655 | 2 | 15302935 | INTRONIC | ENSG00000151779 | Neuroblastoma-amplified gene |
| | | | | | protein |
| rs1550116 | 2 | 24876102 | NON- | CENPO | Centromere protein O (CENP-O) |
| | | | SYNONYMOUS | | (Interphase centromere complex |
| | | | CODING | | protein 36) |
| rs2082881 | 2 | 24891772 | INTRONIC | CENPO | Centromere protein O (CENP-O) |
| | | | | | (Interphase centromere complex |
| | | | | | protein 36) |
| rs1550115 | 2 | 24895124 | INTRONIC | CENPO | Centromere protein O (CENP-O) |
| | | | | | (Interphase centromere complex |
| | | | | | protein 36) |
| rs2302503 | 3 | 37082474 | INTRONIC | LRRFIP2 | Leucine-rich repeat flightless- |
| | | | | | interacting protein 2 (LRR FLII- |
| | | | | | interacting protein 2) |
| rs1040084 | 4 | 54104981 | INTRONIC | LNX1 | E3 ubiquitin-protein ligase LNX |
| | | | | | (EC 6.3.2.—) (Numb-binding |
| | | | | | protein 1) (Ligand of Numb- |
| | | | | | protein X 1) |
| rs1913484 | 4 | 54105081 | INTRONIC | LNX1 | E3 ubiquitin-protein ligase LNX |
| | | | | | (EC 6.3.2.—) (Numb-binding |
| | | | | | protein 1) (Ligand of Numb- |
| | | | | | protein X 1) |
| rs2803073 | 6 | 162882818 | INTRONIC | PARK2 | Parkin (EC 6.3.2.—) (Ubiquitin |
| | | | | | E3 ligase PRKN) (Parkinson |
| | | | | | juvenile disease protein 2) |
| | | | | | (Parkinson disease protein 2) |
| rs10236906 | 7 | 18706195 | INTRONIC | HDAC9 | Histone deacetylase 9 (HD9) |
| | | | | | (HD7B) (HD7) (Histone |
| | | | | | deacetylase-related protein) |
| | | | | | (MEF2-interacting transcription |
| | | | | | repressor MITR) |
| rs9691873 | 7 | 28696534 | INTRONIC | CREB5 | cAMP response element-binding |
| | | | | | protein 5 (CRE-BPa) |
| rs2370220 | 9 | 907667 | INTRONIC | DMRT1 | Doublesex- and mab-3-related |
| | | | | | transcription factor 1 (DM |
| | | | | | domain expressed in testis |
| | | | | | protein 1) |
| rs10814834 | 9 | 4076370 | INTRONIC | GLIS3 | Zinc finger protein GLIS3 (GLI- |
| | | | | | similar 3) (Zinc finger protein |
| | | | | | 515) |
| rs11139349 | 9 | 83433951 | INTRONIC | TLE1 | Transducin-like enhancer protein |
| | | | | | 1 (ESG1) (E(Sp1) homolog) |
| rs7042473 | 9 | 98386391 | INTRONIC | CDC14B, CDC14C | Dual specificity protein |
| | | | | | phosphatase CDC14B (EC |
| | | | | | 3.1.3.48) (EC 3.1.3.16) (CDC14 |
| | | | | | cell division cycle 14 homolog |
| | | | | | B) |
| rs10491723 | 9 | 99967453 | INTRONIC | CORO2A | Coronin-2A (WD repeat- |
| | | | | | containing protein 2) (IR10) |
| rs1411283 | 10 | 27342778 | INTRONIC | ANKRD26 | Ankyrin repeat domain- |
| | | | | | containing protein 26 |
| rs2279519 | 11 | 122982562 | SYNONYMOUS | GRAMD1B | GRAM domain-containing |
| | | | CODING | | protein 1B |
| rs216312 | 12 | 5999245 | INTRONIC | VWF | von Willebrand factor precursor |
| | | | | | (vWF) [Contains: von |
| | | | | | Willebrand antigen 2 (von |
| | | | | | Willebrand antigen II)] |
| rs7968170 | 12 | 16040602 | INTRONIC | DERA | Putative deoxyribose-phosphate |
| | | | | | aldolase (EC 4.1.2.4) |
| | | | | | (Phosphodeoxyriboaldolase) |
| | | | | | (Deoxyriboaldolase) (DERA) |
| rs8012912 | 14 | 69543960 | INTRONIC | SMOC1 | SPARC-related modular |
| | | | | | calcium-binding protein 1 |
| | | | | | precursor (Secreted modular |
| | | | | | calcium-binding protein 1) |
| | | | | | (SMOC-1) |
| rs11158827 | 14 | 69548927 | INTRONIC | SMOC1 | SPARC-related modular |
| | | | | | calcium-binding protein 1 |
| | | | | | precursor (Secreted modular |
| | | | | | calcium-binding protein 1) |
| | | | | | (SMOC-1) |
| rs6576456 | 15 | 23560333 | INTRONIC | ATP10A | Probable phospholipid- |
| | | | | | transporting ATPase VA (EC |
| | | | | | 3.6.3.1) (ATPVA) |
| | | | | | (Aminophospholipid translocase |
| | | | | | VA) |
| rs2899723 | 15 | 65523077 | INTRONIC | IQCH | IQ motif-containing protein H |
| | | | | | (Testis development protein |
| | | | | | NYD-SP5) |
| rs646097 | 17 | 34329857 | 3PRIME UTR | LASP1 | LIM and SH3 domain protein 1 |
| | | | | | (LASP-1) (MLN 50) |
| rs11868362 | 17 | 52853235 | INTRONIC | MSI2 | RNA-binding protein Musashi |
| | | | | | homolog 2 (Musashi-2) |
| rs16943470 | 17 | 54801370 | INTRONIC | YPEL2 | Protein yippee-like 2 |

### Combined Analysis of Rare and Non-Rare Variants

### Methods

It is known that statistical power is greatly affected by allele frequency, so novel methods have arisen for the analysis of rare variants. “Collapsing” or “aggregate” methods allow one to test for association with an accumulation of rare alleles across a locus. The genome-wide marker set was annotated to define gene-based sets, and a Sequence Kernel Association Test (SKAT) was applied, to allow a joint analysis of both common and rare variants, gene by gene (Wu et al, 2011; Ionita-Laza et al, 2013).

Tables were produced listing genes showing at least suggestive significance (p<10⁻⁵).

### Results for Endpoint 1

None of the analyses for Endpoint 1 identified a gene with p<10⁻⁵.

### Results for Endpoint 2

None of the analyses for Endpoint 1 identified a gene with p<10⁻⁵.

### Results for Endpoint 3

Two genes namely LOC100506686 and C15orf61 had p<10⁻⁵ in PGx-GT-Final however, each finding was based upon only a single common marker.

TABLE 38
Association Results with p < 10⁻⁵ for Endpoint 3 in PGx-GT-Final

| | | | | | N Marker |
|---|---|---|---|---|---|
| Gene | p-value | N Marker.All | N Marker.Test | N Marker.Rare | Common |
| LOC100506686 | 8.48e−06 | 1 | 1 | 0 | 1 |
| C15orf61 | 5.29e−06 | 1 | 1 | 0 | 1 |

### Results for Endpoint 4

None of the analyses for Endpoint 4 identified a gene with p<10⁻⁵.

### Results for Endpoint 5

One gene had p<10⁻⁵ in PGx-CN-Final however no rare markers contributed to the result. It backs up a finding described above.

TABLE 39
Association Results with p < 10⁻⁵ for Endpoint 5 in PGx-CN-Final

| | | N Marker | N.Marker | | N Marker |
|---|---|---|---|---|---|
| Gene | p-value | All | Test | N.Marker Rare | Common |
| CENPO | 7.96e−06 | 7 | 7 | 0 | 7 |

### Results for Endpoint 5

One gene, FCER1A had p<10⁻⁵ in PGx-CN-Final. Once again it supports a finding described above however, the joint analysis of all the markers in the gene means that the association now surpasses the threshold for genome-wide significance.

TABLE 40
Association Results with p < 10⁻⁵ for Endpoint 6 in PGx-CN-Final

| | | N Marker | N Marker | N Marker | N Marker |
|---|---|---|---|---|---|
| Gene | p-value | All | Test | Rare | Common |
| FCER1A | 2.65e−08 | 7 | 7 | 1 | 6 |

### Discussion of FCER1A

FCER1A encodes the immunoglobulin epsilon (IgE) Fc receptor subunit alpha. The IgE receptor is the initiator of the allergic response. When two or more high affinity IgE receptors are brought together by allergen-bound IgE molecules, mediators such as histamine are released. The protein encoded by this gene represents the alpha subunit of the receptor.

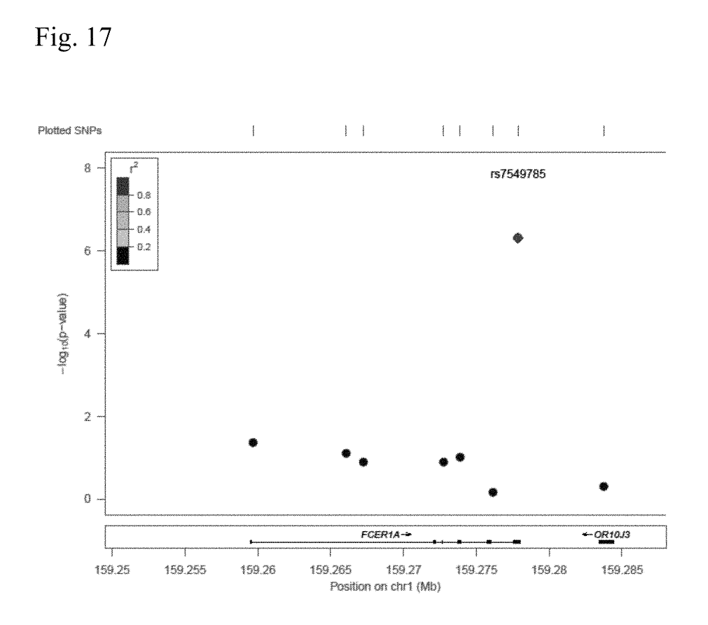

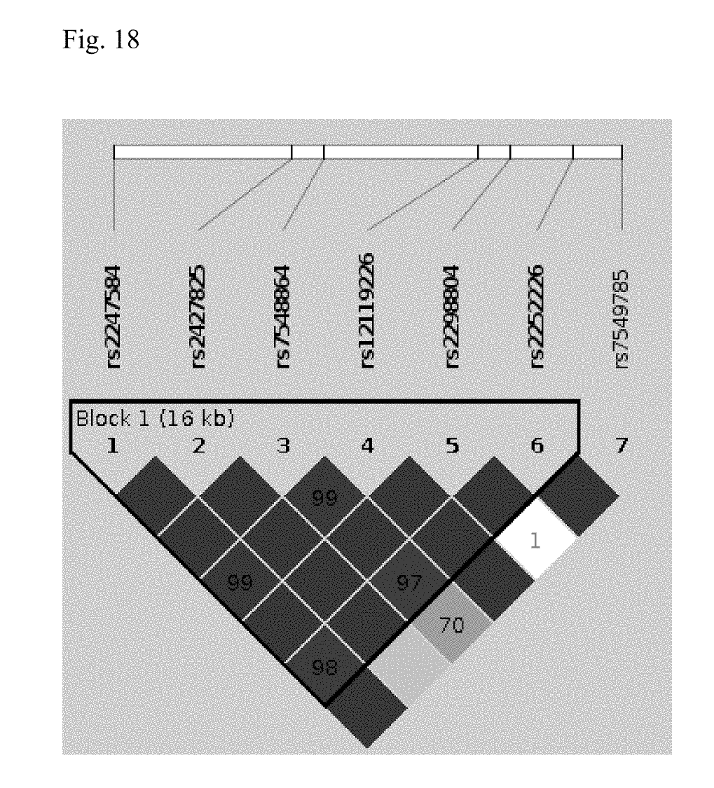

The association between S-loss (Endpoint 6) and FCER1A is driven by a single low-frequency marker in the 3′UTR of the gene. FIG. 17 shows that the association is not shared by flanking markers. FIG. 18 shows that the marker in question, rs7549785 falls outside of a block of linkage disequilibrium which spans the rest of the gene.

Using the cross-tabulation of genotype versus response, given in Table 41, the following preliminary estimates are obtained: sensitivity=24%; specificity=97%; positive predictive value=25%; negative predictive value=93%. The positive predictive value of 25% represents a more than three-fold enrichment compared to the overall rate of S-loss of 7% (80/1095). Unbiased estimates from independent data are required.

The minor allele frequency of the marker, rs7549785 is low, at 2% in PGx-CN-Final, and much higher, 15%, in PGx-nonCN-Final. The association is completely absent from PGx-nonCN-Final with p=0.6143 under an additive model, and p=0.5558 under a dominant model. The overall frequency is 6% in PGx-GT-Final, in which the genotype frequencies show marked departure from Hardy-Weinberg Equilibrium. Due to dilution, the p-values in PGx-GT-Final are p=0.0281 and p=0.0065 respectively. The association, if confirmed, will have arisen due to linkage disequilibrium phenomena (with one or more causal variants) present only in the Southeast Asian group.

TABLE 41
Cross-tabulation of genotype at rs7549785 and response defined by S-loss
at >=24-week follow-up in PGx-CN-Final

| | Non-carrier of A allele | Carrier of A allele | |
|---|---|---|---|
| Non-Response | 982 | 33 | 1015 |
| (S-loss at >=24-week) | | | |
| Positive Response | 69 | 11 | 80 |
| (S-loss at >=24-week) | | | |
| | 1051 | 44 | 1095 |

### Software

Custom-written perl scripts (Wall et al, 1996) were used to reformat the data, select markers for ancestry analysis and produce tables. PLINK version 1.07 (Purcell et al, 2007) was used to perform the genetic QC analyses, to merge study data with HapMap data, and for association analysis. EIGENSOFT 4.0 (Patterson et al, 2006; Price et al, 2006) was used for PCA. R version 2.15.2 (R Core Team, 2012) was used for the production of graphics.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Devlin b, Roeder K (1999). Genomic control for association studies. Biometrics 55(4): 997-1004.
Dienstage J L (2008). Hepatitis B Virus Infection. N Engl J Med 359:1486-1500.
Dunteman G H (1989). Principal Components Analysis. SAGE university papers: Quantitative Applications in the Social Sciences. Series Editor: M S Lewis-Beck. Sage Publications Inc.
Ge D, Fellay J, Thompson A J, Simon J S, Shianna K V, Urban T J, Heinzen E L, Qiu P, Bertelsen A H, Muir A J, Sulkowski M, McHutchison J G, Goldstein D B (2009). Genetic variation in IL28B predicts hepatitis C treatment-induced viral clearance. Nature 161: 399-401.
Guo X, Zhang Y, Li J et al (2011). Strong influence of human leukocyte antigen (HLA)-DP gene variants on development of persistent chronic hepatitis B virus carries in the Han Chinese population. Hepatology 53: 422-8.
The International HapMap Consortium (2003). The International HapMap Project. Nature 426: 789-796.
The International HapMap Consortium (2005). A Haplotype Map of the Human Genome. Nature 437: 1299-1320.
The International HapMap Consortium (2007). A second generation human haplotype map of over 3.1 million SNPs. Nature 449:851-861.
Ionita-Laza L, Lee S, Makarov V, Buxbaum J, Lin X (2013). Sequence kernel association tests for the combined effect of rare and common variants. Am J Hum Genet 92: 841-853.
Liu L, Li Y, Zhang S, Yu D, Zhu M (2012). Hepatitis B virus X protein mutant upregulates CENP-A expression in hepatoma cells. Oncol Rep. 27(1): 168-73.
Liu Y, Wang X, Li S, Hu H, Zhang D, Hu P, Yang Y, Ren H (2014). The role of von Willebrand factor as a biomarker of tumour development in hepatitis B virus-associated human hepatocellular carcinoma: a quantitative proteomic based study. J Proteomics 106:99-112.
Kamatani Y, Wattanapokayakit X, Ochi H et al (2009). a genome-wide association study identifies variants in the HLA-DP locus associated with chronic hepatitis B in Asians. Nat Genet 41: 591-595.
Muller U, Steinhoff U, Reis LF, et al (1994). Functional role of type I and type II interferons in antiviral defence. Science 264: 1918-21.
Patterson N, Price A L, Reich D. Population structure and Eigenanalysis (2006). PLoS Genet. 2: el90.
Price AL, Patterson N J, Plenge R M, Weinblatt M E, Shadick N A, Reich D (2006). Principal components analysis corrects for stratification in genome-wide association studies. Nat Genet. 38:904.
Purcell S, Neale B, Todd-Brown K, Thomas L, Ferreira MAR, Bender D, Maller J, Sklar P, de Bakker P I W, Daly M J, Sham PC (2007). PLINK: a toolset for whole-genome association and population-based linkage analysis. Am J Hum Genet 81 (3):559-575.
R Core Team (2012). R: A language and environment for statistical computing. R Foundation for statistical computing, Vienna, Austria. ISBN 3-900051-07-0, URL http://www.R-project.org/)
Tanaka Y, Nishida N, Sugiyama M, Kurosaki M, Matsuura K, Sakamoto N, Nakagawa M, Korenaga M, Hino K, Hige S, Ito Y, Mita E, Tanaka E, Mochida S, Murawaki Y, Honda M, Sakai A, Hiasa Y, Nishiguchi S, Koike A, Sakaida I, Imamura M, Ito K, Yano K, Masaki N, Sugauchi F, Izumi N, Tokunaga K, Mizokami M (2009). Genome-wide association of IL28B with response to pegylated interferon-alpha and ribavirin therapy for chronic hepatitis C. Nat Genet 41(10): 1105-1109.
Tang R, Kong F, Hu L, You H, Zhang P, Du W, Zheng K (2012). Role of hepatitis B virus X protein in regulatin LIM and SH3 protein 1 (LASP-1) expression to mediate proliferation and migration of hepatoma cells. Virol J 9: 163.
Wall L, Christiansen T, Schwartz R L (1996). Programming Perl (2^{nd} Edition). O'Reilly and Associates Inc (USA).
Whitlock M C (2005). Combining probability from independent tests: the weighted Z method is superior to Fisher's approach. J Evol Biol 18: 1368-1373.
WHO Fact Sheet No. 204; Revised August 2008.
Wu M C, Lee S, Cai T, Li Y, Boehnke M, Lin X (2011). Rare-variant association testing for sequence data with the sequence kernal association test. Am J Hum Genet 89:82-93.

1. A method of identifying a patient who may benefit from treatment with an anti-HBV therapy comprising an interferon, the method comprising:
determining the presence of a single nucleotide polymorphism in gene FCER1A on chromosome 1 in a sample obtained from the patient, wherein the presence of at least one A allele at rs7549785 indicates that the patient may benefit from the treatment with the anti-HBV treatment.

2. A method of predicting responsiveness of a patient suffering from an HBV infection to treatment with an anti-HBV treatment comprising an interferon, the method comprising:
determining the presence of a single nucleotide polymorphism in gene FCER1A on chromosome 1 in a sample obtained from the patient, wherein the presence of at least one A allele at rs7549785 indicates that the patient is more likely to be responsive to treatment with the anti-HBV treatment.

3. A method for determining the likelihood that a patient with an HBV infection will exhibit benefit from an anti-HBV treatment comprising an interferon, the method comprising:
determining the presence of a single nucleotide polymorphism in gene FCER1A on chromosome 1 in a sample obtained from the patient, wherein the presence of at least one A allele at rs7549785 indicates that the patient has increased likelihood of benefit from the anti-HBV treatment.

4. A method for optimizing the therapeutic efficacy of an anti-HBV treatment comprising an interferon, the method comprising:
determining the presence of a single nucleotide polymorphism in gene FCER1A on chromosome 1 in a sample obtained from the patient, wherein the presence of at least one A allele at rs7549785 indicates that the patient has increased likelihood of benefit from the anti-HBV treatment.

5. A method for treating an HBV infection in a patient, the method comprising:
(i) determining the presence of at least one A allele at rs7549785 in gene FCER1A on chromosome 1 in a sample obtained from the patient and
(ii) administering an effective amount of an anti-HBV treatment comprising an interferon to said patient, whereby the HBV infection is treated.

6. A method for predicting S-loss at >=24-week follow-up of treatment (responders vs. non-responders) of a patient infected with HBV to interferon treatment comprising:
providing a sample from said human subject, detecting the presence of a single nucleotide polymorphism in gene FCER1A on chromosome 1 and determining that said patient has a high response rate to interferon treatment measured as S-loss at >=24-week follow-up of treatment (responders vs. non-responders) if at least one A allele at rs7549785 is present.

7. The method of claim 1, wherein the interferon is selected from the group consisting of peginterferon alfa-2a, peginterferon alfa-2b, interferon alfa-2a and interferon alfa-2b.

8. The method of claim 7, wherein the interferon is a peginterferon alfa-2a conjugate having the formula: 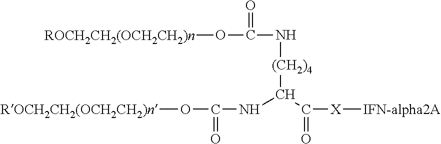
wherein R and R′ are methyl, X is NH, and n and n′ are individually or both either 420 or 520.
